(19) United States
(12) Patent Application Publication
(10) Pub. No.: US 2014/0130199 A1
(43) Pub. Date: May 8, 2014

(54) COMMON WHEAT, PLANTS OR PARTS THEREOF HAVING PARTIALLY OR FULLY MULTIPLIED GENOME, HYBRIDS AND PRODUCTS THEREOF AND METHODS OF GENERATING AND USING SAME

(71) Applicants: Amit Avidov, Kiryat-Tivon (IL); Alon Lerner, Moshav Sharona (IL); Itamar Lupo, Moshav Elad (IL)

(72) Inventors: Amit Avidov, Kiryat-Tivon (IL); Alon Lerner, Moshav Sharona (IL); Itamar Lupo, Moshav Elad (IL)

(73) Assignee: Kaiima Bio Agritech Ltd., Kfar-Tavor (IL)

(21) Appl. No.: 14/128,706

(22) PCT Filed: Jun. 19, 2012
(86) PCT No.: PCT/IL2012/050211

§ 371 Date: Dec. 23, 2013

Related U.S. Application Data
(60) Provisional application No. 61/500,183, filed on Jun. 23, 2011.

(51) Int. Cl. A01H 5/10

(52) U.S. Cl. CPC A01H 5/10
USPC 800/260; 800/320.3; 435/419; 800/276; 426/518

(57) ABSTRACT

A common wheat plant is provided. The plant has a partially or fully multiplied genome being at least as fertile as a hexaploid common wheat (Triticum aestivum L.) plant isogenic to said genomically multiplied common wheat plant when grown under the same conditions. Also provided are hybrids, products and methods of generating same.

### FIELD AND BACKGROUND OF THE INVENTION

The present invention, in some embodiments thereof, relates to common wheat plants or parts thereof having partially or fully multiplied genome, hybrids and products thereof and methods of generating and using same.

Wheat of the Triticum spp., also known as bread wheat or common wheat (Triticum aestivum L.) is a grass, originally from the Fertile Crescent region of the Near East, but now cultivated worldwide. In 2007 world production of wheat was 607 million tons, making it the third most-produced cereal after maize (784 million tons) and rice (651 million tons). Globally, wheat is the leading source of vegetable protein in human food, having a higher protein content than either maize (corn) or rice, the other major cereals. In terms of total production tonnages used for food, it is currently second to rice as the main human food crop, and ahead of maize, after allowing for maize's more extensive use in animal feeds.

Wheat was a key factor enabling the emergence of city-based societies at the start of civilization because it was one of the first crops that could be easily cultivated on a large scale, and had the additional advantage of yielding a harvest that provides long-term storage of food. Wheat is a factor in contributing to city-states in the Fertile Crescent including the Babylonian and Assyrian empires. Wheat grain is a staple food used to make flour for leavened, flat and steamed breads, biscuits, cookies, cakes, breakfast cereal, pasta, noodles, couscous and for fermentation to make beer, other alcoholic beverages, or biofuel.

Wheat is planted to a limited extent as a forage crop for livestock, and its straw can be used as a construction material for roofing thatch. The whole grain can be milled to leave just the endosperm for white flour. The products of this are bran and germ. The whole grain is a concentrated source of vitamins, minerals, and protein, while the refined grain is mostly starch.

Demand for wheat is increasing. As the large populations in Asia become wealthier, they are consuming more wheat products and a lesser proportion of traditional foods such as rice. Likewise, more meat products produced from grain fed animals are being consumed. Grain is also being increasingly diverted from the food supply and into ethanol production to replace fossil fuels.

Wheat breeding for improved productivity has been noticeably successful during the second half of the 20^{th} century. However, wheat yield has to be further increased in order to meet the constantly increasing demand of a still rapidly increasing population whilst facing a reduction, or at least a lack of expansion of arable land if environmental sustainability is to be achieved.

### RELATED ART

U.S Patent Application Number: 20030005479
Lazareva et al. Cell Biol. 2000; 14(2):163-72.
Siddiqui Basic Life Sci. 1976 Mar. 1-7; 8:97-102.
Fragata Experientia. 1970 Jan. 15; 26(1):104-6.
Tang P S, Loo W S. Science. 1940 Mar. 1; 91(2357):222.
Dvorak 1973 Can. J. Genet. Cytol. 15:205-214
Zhang et al. J. 2008 Appl. Genet. 49(1):41-44
WO2009/060453
WO2009/060455

### SUMMARY OF THE INVENTION

According to an aspect of some embodiments of the present invention there is provided a common wheat plant having a partially or fully multiplied genome being at least as fertile as a hexaploid common wheat (Triticum aestivum L.) plant isogenic to the genomically multiplied common wheat plant when grown under the same conditions.

According to an aspect of some embodiments of the present invention there is provided a hybrid plant having as a parental ancestor the above-plant.

According to an aspect of some embodiments of the present invention there is provided a hybrid common wheat plant having a partially or fully multiplied genome.

According to an aspect of some embodiments of the present invention there is provided a planted field comprising any of the above plants.

According to an aspect of some embodiments of the present invention there is provided a sown field comprising seeds of any of the above plants.

According to some embodiments of the invention, the plant is non-transgenic.

According to some embodiments of the invention, the plant has a spike number at least as similar to that of the hexaploid common wheat (Triticum aestivum L.) plant under the same developmental stage and growth conditions.

According to some embodiments of the invention, the plant has a spikelet number at least as similar to that of the hexaploid common wheat (Triticum aestivum L.) plant under the same developmental stage and growth conditions.

According to some embodiments of the invention, the plant has a spike length at least as similar to that of the hexaploid common wheat (Triticum aestivum L.) plant under the same developmental stage and growth conditions.

According to some embodiments of the invention, the plant has a spike width at least as similar to that of the hexaploid common wheat (Triticum aestivum L.) plant under the same developmental stage and growth conditions.

According to some embodiments of the invention, the plant has a spike internode number at least as similar to that of the hexaploid common wheat (Triticum aestivum L.) plant under the same developmental stage and growth conditions.

According to some embodiments of the invention, the plant has a grain protein content at least as similar to that of the hexaploid common wheat (Triticum aestivum L.) plant under the same developmental stage and growth conditions.

According to some embodiments of the invention, the plant has a dry matter content at least as similar to that of the hexaploid common wheat (Triticum aestivum L.) plant under the same developmental stage and growth conditions.

According to some embodiments of the invention, the plant has a grain yield per growth area at least as similar to that of the hexaploid common wheat (Triticum aestivum L.) plant under the same developmental stage and growth conditions.

According to some embodiments of the invention, the plant has a grain number per spikelet ratio at least as similar to that of the hexaploid common wheat (Triticum aestivum L.) plant under the same developmental stage and growth conditions.

According to some embodiments of the invention, the plant has a total grain number per plant ratio at least as similar to that of the hexaploid common wheat (Triticum aestivum L.) plant under the same developmental stage and growth conditions.

According to some embodiments of the invention, the plant has a grain weight at least as similar to that of the hexaploid common wheat (Triticum aestivum L.) plant under the same developmental stage and growth conditions.

According to some embodiments of the invention, the plant has a grain yield per plant at least as similar to that of the hexaploid common wheat (Triticum aestivum L.) plant under the same developmental stage and growth conditions.

According to some embodiments of the invention, the plant has a rust tolerance higher than that of the hexaploid common wheat (Triticum aestivum L.) plant under the same developmental stage and growth conditions.

According to some embodiments of the invention, the plant has a total plant length similar or lower than that of the hexaploid common wheat (Triticum aestivum L.) plant under the same developmental stage and growth conditions.

According to some embodiments of the invention, the fertility is determined by at least one of:

number of seeds per plant;

seed set assay;

gamete fertility assay; and

acetocarmine pollen staining

According to some embodiments of the invention, the plant is a dodecaploid.

According to some embodiments of the invention, the plant is an octaploid.

According to some embodiments of the invention, the plant is a decaploid.

According to some embodiments of the invention, the plant is capable of cross-breeding with a hexaploid or a tetraploid wheat.

According to some embodiments of the invention, the wheat is a Durum wheat (Triticum durum).

According to some embodiments of the invention, the hybrid plant has a Durum wheat as a second parental ancestor.

According to some embodiments of the invention, the plant is an autopolyploid.

According to an aspect of some embodiments of the present invention there is provided a plant part of the common wheat plant.

According to an aspect of some embodiments of the present invention there is provided a processed product of the plant or plant part.

According to some embodiments of the invention, the processed product is selected from the group consisting of food, feed, construction material and biofuel.

According to some embodiments of the invention, the food or feed is selected from the group consisting of breads, biscuits, cookies, cakes, pastries, snacks, breakfast cereal, pasta, noodles, couscous, beer and alcoholic beverages.

According to an aspect of some embodiments of the present invention there is provided a meal produced from the plant or plant part.

According to some embodiments of the invention, the plant part is a seed.

According to an aspect of some embodiments of the present invention there is provided an isolated regenerable cell of the common wheat plant.

According to some embodiments of the present invention, the cell exhibits genomic stability for at least 5 passages in culture.

According to some embodiments of the present invention, the cell is from a mertistem, a pollen, a leaf, a root, a root tip, an anther, a pistil, a flower, a seed, a grain or a stem.

According to an aspect of some embodiments of the present invention there is provided a tissue culture comprising the regenerable cells.

According to an aspect of some embodiments of the present invention there is provided a method of producing common wheat seeds, comprising self-breeding or cross-breeding the plant.

According to an aspect of some embodiments of the present invention there is provided a method of developing a hybrid plant using plant breeding techniques, the method comprising using the plant as a source of breeding material for self-breeding and/or cross-breeding.

According to an aspect of some embodiments of the present invention there is provided a method of producing common wheat meal, the method comprising:
(a) harvesting grains of the common Triticum aestivum L. plant or plant part; and
(b) processing the grains so as to produce the common Triticum aestivum L. meal.

According to an aspect of some embodiments of the present invention there is provided a method of generating a common wheat seed having a partially or fully multiplied genome, the method comprising contacting a common wheat (Triticum aestivum L.) seed with a G2/M cell cycle inhibitor under a transiently applied magnetic field thereby generating the common wheat seed having a partially or fully multiplied genome.

According to some embodiments of the invention, the G2/M cell cycle inhibitor comprises a microtubule polymerization inhibitor.

According to some embodiments of the invention, the microtubule polymerization inhibitor is selected from the group consisting of colchicine, nocodazole, oryzaline, trifluraline and vinblastine sulphate.

According to some embodiments of the invention, the method comprises sonicating the seed prior to contacting.

According to an aspect of some embodiments of the present invention there is provided a sample of representative seeds of a common wheat plant having a partially or fully multiplied genome being at least as fertile as a hexaploid common wheat (Triticum aestivum L.) plant isogenic to the genomically multiplied common wheat (Triticum aestivum L.) plant when grown under the same conditions, wherein the sample has been deposited under the Budapest Treaty at the NCIMB under NCIMB 41972.

According to an aspect of some embodiments of the present invention there is provided a sample of representative seeds of a common wheat plant having a partially or fully multiplied genome being at least as fertile as a hexaploid common wheat (Triticum aestivum L.) plant isogenic to the genomically multiplied common wheat plant when grown under the same conditions, wherein the sample of the common wheat plant having the partially or fully multiplied genome has been deposited under the Budapest Treaty at the NCIMB under NCIMB 41972.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below in case of conflict, the patent specification, including definitions, will control in addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention in this regard, the description taken with the drawings makes apparent to those skilled in the art how embodiments of the invention may be practiced.

In the drawings:

FIG. 1 is an image of an R-2010-1 polyploidy stable line (336. See FACS results in Table 5) compared with the isogenic R-2010-1 normal ploidy line.

FIG. 2 is an image of the HF1 hybrid using line H-2010-4 demonstrating high heterosis in winter bread wheat.

FIG. 3 is an image of the HF1 hybrid using line R-2010-1 demonstrating high heterosis in winter bread wheat.

FIG. 4 is a bar graph demonstrating phenotypic parameters of the hybrid wheat W2(620) and the parental female plant.

FIG. 5 is a bar graph demonstrating phenotypic parameters of the parental male plant used in the hybrid W2(620) and its 12N isogenic male line.

FIG. 6 is a bar graph demonstrating phenotypic parameters of the hybrid wheat W15(659) and the parental female plant.

FIG. 7 is a bar graph demonstrating phenotypic parameters of the parental male plant used in the hybrid W15(659) and its 12N isogenic male line.

FIG. 8 is a bar graph demonstrating phenotypic parameters of the hybrid wheat W16(648) and the parental female plant.

FIG. 9 is a bar graph demonstrating phenotypic parameters of the parental male plant used in the hybrid W16(648) and its 8N isogenic male line.

FIG. 10 is a bar graph demonstrating phenotypic parameters of the hybrid wheat W17(650) and the parental female plant.

FIG. 11 is a bar graph demonstrating phenotypic parameters of the parental male plant used in the hybrid W17(650) and its 10N isogenic male line.

FIG. 12 is a bar graph demonstrating phenotypic parameters of the hybrid wheat W18(669) and the parental female plant.

FIG. 13 is a bar graph demonstrating phenotypic parameters of the parental male plant used in the hybrid W18(669) and its 12N isogenic male line.

FIG. 14 is a bar graph demonstrating phenotypic parameters of the hybrid wheat W19(681) and the parental female plant.

FIG. 15 is a bar graph demonstrating phenotypic parameters of the parental male plant used in the hybrid W19(681) and its 12N isogenic male line.

FIG. 16 is a graph showing FACS analysis of DNA content in hexaploid common wheat “3-control” line. The left peak of PI-A value is 320, (each unit marks 100 units). The right peak presents the cell cycle.

FIG. 17 is a graph showing FACS analysis of DNA content in induced polyploid “3-20-1020-1” line. The peak of PI-A value is 540 (each unit marks 100 units), which confirms that the amount of DNA represents a partially or fully multiplied genome.

FIG. 18 is a graph showing FACS analysis of DNA content of induced polyploid “3-20-1030-1” line. The peak of PI-A value is 480 (each unit marks 100 units), which confirms that the amount of DNA represents a partially or fully multiplied genome.

FIG. 19 is a graph showing FACS analysis of DNA content of induced polyploid “5-control” line. The left peak of PI-A value is 320, (each unit marks 100 units). The right peak presents the cell cycle.

FIG. 20 is a graph showing FACS analysis of DNA content of induced polyploid “5-1226-1” line. The peak of PI-A value is 440 (each unit marks 100 units), which confirms that the amount of DNA represents a partially or fully multiplied genome.

FIG. 21 is a graph showing FACS analysis of DNA content of induced polyploid “15-control” line. The left peak of PI-A value is 300, (each unit marks 100 units). The right peak presents the cell cycle.

FIG. 22 is a graph showing FACS analysis of DNA content of induced polyploid “15-94” line. The peak of PI-A value is 420 (each unit marks 100 units), which confirms that the amount of DNA represents a partially or fully multiplied genome.

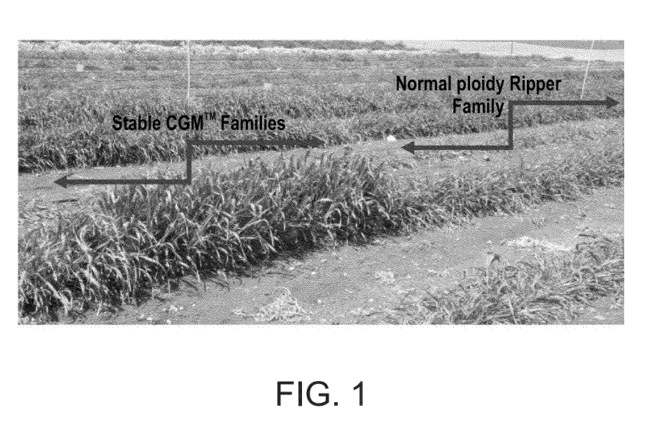

### DESCRIPTION OF SPECIFIC EMBODIMENTS OF THE INVENTION

The present invention, in some embodiments thereof, relates to common wheat plants or parts thereof having partially or fully multiplied genome, hybrids and products thereof and methods of generating and using same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Induced polyploidy has been suggested for increasing plant yields. To date, however, induced polyploidy has been successfully achieved for only a few plant species.

The present inventors have now designed a novel procedure for induced genome multiplication in common wheat (Triticum aestivum L.) that results in plants which are genomically stable and fertile. The induced polyploid plants are devoid of undesired genomic mutations and are characterized by stronger vigor and higher total plant yield than that of the isogenic progenitor plant having a hexaploid genome (see Table 5, below). These new traits may contribute to better climate adaptability and higher tolerance to biotic and abiotic stress. Furthermore, hybrid wheat seeds generated by pollen sterilization using the induced polyploid plants of the present invention may increase global wheat yield in few tens percent due to heterosis expression. In addition, the induced polyploid plant of some embodiments of the invention exhibits a similar or better fertility compared to that of the isogenic hexaploid progenitor plant already from early generations (e.g., first, second, third or fourth) following genome multiplication, negating the need for further breeding in order to improve fertility.

Thus, according to an aspect of the invention there is provided a common wheat plant having a partially or fully multiplied genome being at least as fertile as a hexaploid common wheat (Triticum aestivum L.) plant isogenic to said genomically multiplied common wheat plant when grown under the same conditions.

As used herein, the term “common wheat” (also referred to herein as “bread wheat”) refers to the Triticum aestivum L. species of the Triticum genus. The genome is an allohexaploid, an allopolyploid with six sets of chromosomes, two sets from each of three different species, AABBDD (2n−6x=42) in its non-multiplied form (euploid). Thus, according to a specific embodiment, the genetic composition of the non-multiplied (i.e., progenitor) plant is AABBDD genome of common wheat.

According to a specific embodiment the common wheat may be naturally occurring or a synthetic wheat.

“A plant” refers to a whole plant or portions thereof (e.g., seeds, grains, stems, fruit, leaves, flowers, tissues etc.), processed or non-processed (e.g., seeds, meal, dry tissue, cake etc.), regenerable tissue culture or cells isolated therefrom. According to some embodiments, the term plant as used herein also refers to hybrids having one of the induced polyploid plants as at least one of its ancestors, as will be further defined and explained hereinbelow.

As used herein “partially or fully multiplied genome” refers to an addition of at least one chromosome, an ancestral genome set (e.g., AA, BB or DD), a mixed ancestral set of chromosomes (e.g., AB, BD, AD) or more (e.g., 2 sets), say a full multiplication of the genome that results in a dodecaploid plant (12N) or more.

The plant of some embodiments of the invention is also referred to herein as “induced polyploid” plant.

According to a specific embodiment, the induced polyploid plant is 8N.

According to a specific embodiment, the induced polyploid plant is 10N.

According to a specific embodiment, the induced polyploid plant is 12N.

According to a specific embodiment, the induced polyploid plant is 14N.

According to a specific embodiment, the induced polyploid plant is 16N.

According to a specific embodiment, the induced polyploid plant is 18N.

According to a specific embodiment, the induced polyploid plant is 24N.

According to a specific embodiment, the induced polyploid plant is 26N.

According to a specific embodiment, the induced polyploid plant is 28N.

According to a specific embodiment, the induced polyploid plant is 30N.

According to a specific embodiment, the induced polyploid plant is 32N.

According to a specific embodiment, the induced polyploid plant is 34N.

According to a specific embodiment, the induced polyploid plant is 36N.

According to a specific embodiment, the induced polyploid plant is not a genomically multiplied haploid plant.

As mentioned, the induced polyploid is at least as fertile as the hexaploid common wheat progenitor plant isogenic to the genomically multiplied common wheat when grown under the same (identical) conditions.

As used herein the term “fertile” refers to the ability to reproduce sexually. Fertility can be assayed using methods which are well known in the art. Alternatively, fertility is defined as the ability to set seeds. The following parameters may be assayed in order to determine fertility: the number of seeds (grains); seed set assay, gamete fertility may be determined by pollen germination such as on a sucrose substrate; and alternatively or additionally acetocarmine staining, whereby a fertile pollen is stained.

As used herein the term “stable” or “genomic stability” refers to the number of chromosomes or chromosome copies, which remains constant through several generations, while the plant exhibits no substantial decline in at least one of the following parameters: yield, fertility, biomass, vigor. According to a specific embodiment, stability is defined as producing a true to type offspring, keeping the variety strong and consistent.

According to a specific embodiment, the plant exhibits genomic stability for at least 2, 3, 5, 10 or more passages in culture or generations.

According to an embodiment of the invention, the genomically multiplied plant is isogenic to the source plant, namely the hexaploid wheat. The genomically multiplied plant has substantially the same genomic composition as the hexaploid plant in quality but not in quantity.

According to some embodiments of the present invention, a mature genomically multiplied plant has at least about the same (+/−10%) number of seeds as it's isogenic hexaploid progenitor grown under the same conditions; alternatively or additionally the genomically multiplied plant has at least 90% fertile pollen that are stained by acetocarmine; and alternatively or additionally at least 90% of seeds germinate on sucrose. As can be seen from Table 5 below, octaploid, decaploid and dodecaploid plants generated according to the present teachings have total yield/plant which is higher by at least 25% than that of the isogenic progenitor plant. According to a specific embodiment, yield is measured using the following formula:

Yield per plant=total grain number/plant×grain weight

Comparison assays done for characterizing traits (e.g., fertility, yield, biomass and vigor) of the genomically multiplied plants of the present invention are typically effected in comparison to it's isogenic progenitor (hereinafter, “the hexaploid progenitor plant”) when both are being of the same developmental stage and both are grown under the same growth conditions.

According to a specific embodiment, the genomically multiplied plant is characterized by a spike number at least as similar to that of the hexaploid common wheat (Triticum aestivum L.) isogenic progenitor plant of the same developmental stage and grown under the same growth conditions. According to a specific embodiment the spike number is higher by 2%, 3%, 4%, 5%, 6%, 7%, 8%, 9%, 10% or even more 15% or 20%. According to a specific embodiment the spike number is higher by 0.5-20%, 0.5-15% or 1-20% higher than that of the isogenic progenitor plant of the same developmental stage and grown under the same growth conditions.

According to a specific embodiment, the genomically multiplied plant is characterized by a spikelet number at least as similar to that of the hexaploid common wheat (Triticum aestivum L.) isogenic progenitor plant of the same developmental stage and grown under the same growth conditions. According to a specific embodiment the a spikelet number is higher by 2%, 3%, 4%, 5%, 6%, 7%, 8%, 9%, 10% or even more 15% or 20%. According to a specific embodiment the spikelet number is higher by 0.5-20%, 0.5-15% or 1-20% higher than that of the isogenic progenitor plant of the same developmental stage and grown under the same growth conditions.

According to a specific embodiment, the genomically multiplied plant is characterized by a spike length at least as similar to that of the hexaploid common wheat (Triticum aestivum L.) isogenic progenitor plant of the same developmental stage and grown under the same growth conditions. According to a specific embodiment the spike length is higher by 2%, 3%, 4%, 5%, 6%, 7%, 8%, 9%, 10% or even more 15 or 20%. According to a specific embodiment the spike length is higher by 0.5-20%, 0.5-15% or 1-13% higher than that of the isogenic progenitor plant of the same developmental stage and grown under the same growth conditions.

According to a specific embodiment, the genomically multiplied plant is characterized by grain number per spikelet at least as similar to that of the hexaploid common wheat (Triticum aestivum L.) isogenic progenitor plant of the same developmental stage and grown under the same growth conditions. According to a specific embodiment the grain number per spikelet is higher by 2%, 3%, 4%, 5%, 6%, 7%, 8%, 9%, 10% or even more 15% or 20%. According to a specific embodiment the grain number per spikelet is higher or lower by about 0-10% of that of the isogenic progenitor plant of the same developmental stage and grown under the same growth conditions.

According to a specific embodiment, the genomically multiplied plant is characterized by spike width at least as similar to that of the hexaploid common wheat (Triticum aestivum L.) isogenic progenitor plant of the same developmental stage and grown under the same growth conditions. According to a specific embodiment the spike width is higher or lower by about 0-10% of that of the isogenic progenitor plant of the same developmental stage and grown under the same growth conditions.

According to a specific embodiment, the genomically multiplied plant is characterized by spike internode number at least as similar to that of the hexaploid common wheat (Triticum aestivum L.) isogenic progenitor plant of the same developmental stage and grown under the same growth conditions. According to a specific embodiment the spike internode number is higher or lower by about 0-10% of that of the isogenic progenitor plant of the same developmental stage and grown under the same growth conditions.

According to a specific embodiment, the genomically multiplied plant is characterized by grain protein content at least as similar to that of the hexaploid common wheat (Triticum aestivum L.) isogenic progenitor plant of the same developmental stage and grown under the same growth conditions. According to a specific embodiment the grain protein content is higher or lower by about 0-20% of that of the isogenic progenitor plant of the same developmental stage and grown under the same growth conditions.

According to a specific embodiment, the genomically multiplied plant is characterized by a dry matter content at least as similar to that of the hexaploid common wheat (Triticum aestivum L.) isogenic progenitor plant of the same developmental stage and grown under the same growth conditions. According to a specific embodiment the dry matter content is higher by 2%, 3%, 4%, 5%, 6%, 7%, 8%, 9%, 10% or even more 15%, 20%, 30%, 40%, 50%, 60%, 70%, 80% or 100%. According to a specific embodiment the grain weight is higher by 1-100%, 1-20%, 5-50% or 5-80% than that of the isogenic progenitor plant of the same developmental stage and grown under the same growth conditions.

According to a specific embodiment, the genomically multiplied plant is characterized by a grain yield per growth area at least as similar to that of the hexaploid common wheat (Triticum aestivum L.) isogenic progenitor plant of the same developmental stage and grown under the same growth conditions. According to a specific embodiment the grain yield per growth area is higher by 10%, 15%, 20%, 25%, 30%, 35%, 40%, 45%, 50% or even more 80%, 90%, 100%, 200, %, 250%, 300%, 400% or 500%. According to a specific embodiment the grain yield per growth area is higher by 0.1-5, 0.3-5, 0.4-2.5, 1-5, 2-3 or 2-2.5 fold than that of the isogenic progenitor plant of the same developmental stage and grown under the same growth conditions.

According to a specific embodiment, the genomically multiplied plant is characterized by grain weight at least as similar to that of the hexaploid common wheat (Triticum aestivum L.) isogenic progenitor plant of the same developmental stage and grown under the same growth conditions. According to a specific embodiment the grain weight is higher by 2%, 3%, 4%, 5%, 6%, 7%, 8%, 9%, 10% or even more 15% or 20%. According to a specific embodiment the grain weight is higher by 1-50%, 1-20% or 5-20% than that of the isogenic progenitor plant of the same developmental stage and grown under the same growth conditions.

According to a specific embodiment, the genomically multiplied plant is characterized by a total grain number per plant at least as similar to that of the hexaploid common wheat (Triticum aestivum L.) isogenic progenitor plant of the same developmental stage and grown under the same growth conditions. According to a specific embodiment the total grain number per plant is higher by 10%, 15%, 20%, 25%, 30%, 35%, 40%, 45%, 50% or even more 80%, 90%, 100%, 200, %, 250%, 300%, 400% or 500%. According to a specific embodiment the total grain number per plant is higher by 0.1-5, 0.3-5, 0.4-2.5, 1-5, 2-3 or 2-2.5 fold than that of the isogenic progenitor plant of the same developmental stage and grown under the same growth conditions.

According to a specific embodiment, the genomically multiplied plant is characterized by a grain yield per plant at least as similar to that of the hexaploid common wheat (Triticum aestivum L.) isogenic progenitor plant of the same developmental stage and grown under the same growth conditions. According to a specific embodiment the grain yield per plant is higher by 10%, 15%, 20%, 25%, 30%, 35%, 40%, 45%, 50% or even more 80%, 90%, 100%, 200, %, 250%, 300%, 400% or 500%. According to a specific embodiment the total grain yield per plant is higher by 0.1-5, 0.3-5, 0.4-2.5, 1-5, 2-3 or 2-2.5 fold than that of the isogenic progenitor plant of the same developmental stage and grown under the same growth conditions.

According to a specific embodiment, the genomically multiplied plant is characterized by a tiller number at least as similar to that of the hexaploid common wheat (Triticum aestivum L.) isogenic progenitor plant of the same developmental stage and grown under the same growth conditions. According to a specific embodiment the tiller number is higher by 10%, 15%, 20%, 25%, 30%, 35%, 40%, 45%, 50% or even more 80%, 90%, 100%, 200, %, 250%, 300%, 400% or 500%. According to a specific embodiment the tiller number is higher by 0.1-5, 0.3-5, 0.4-2.5, 1-5, 2-3 or 2-2.5 fold than that of the isogenic progenitor plant of the same developmental stage and grown under the same growth conditions.

According to a specific embodiment, the genomically multiplied plant is characterized by a rust tolerance at least as similar to that of the hexaploid common wheat (Triticum aestivum L.) isogenic progenitor plant of the same developmental stage and grown under the same growth conditions. According to a specific embodiment the rust tolerance is higher by 2%, 3%, 4%, 5%, 6%, 7%, 8%, 9%, 10% or even more 15%, 20%, 30% or 40%. According to a specific embodiment the rust tolerance is higher by 1-50%, 1-20% or 5-20% than that of the isogenic progenitor plant of the same developmental stage and grown under the same growth conditions.

Interestingly, the plants of the invention are characterized by an above ground plant length (or height) that is similar or shorter or higher than that of the isogenic progenitor plant of the same developmental stage and grown under the same growth conditions (see Table 5 below). According to a specific embodiment the plant length is shorter or higher by 2%, 3%, 4%, 5%, 6%, 7%, 8%, 9%, 10% or even more 15% or 20%. According to a specific embodiment the above ground plant length (or height) is higher or lower by about 0-20% of that of the isogenic progenitor plant of the same developmental stage and grown under the same growth conditions.

According to a specific embodiment, the plant is non-transgenic.

According to another embodiment, the plant is transgenic for instance by expressing a heterologous gene conferring pest resistance or morphological traits for cultivation, as further described herein below.

Genomically multiplied plant seeds of the present invention can be generated using an improved method of colchicination, as described below.

Thus, according to an aspect of the invention, there is provided a method of generating a common wheat seed having a partially or fully multiplied genome, the method comprising contacting a common wheat (Triticum aestivum L.) seed with a G2/M cell cycle inhibitor under a transiently applied magnetic field, thereby generating the common wheat seed having a partially or fully multiplied genome.

Typically, the G2/M cycle inhibitor comprises a microtubule polymerization inhibitor.

Examples of microtubule cycle inhibitors include, but are not limited colchicine, colcemid, trifluralin, oryzalin, benzimidazole carbamates (e.g. nocodazole, oncodazole, mebendazole, R 17934, MBC), o-isopropyl N-phenyl carbamate, chloroisopropyl N-phenyl carbamate, amiprophos-methyl, taxol, vinblastine, griseofulvin, caffeine, bis-ANS, maytansine, vinbalstine, vinblastine sulphate and podophyllotoxin.

The G2/M inhibitor is comprised in a treatment solution which may include additional active ingredients such as antioxidants, detergents and histones.

While treating the seeds with a treatment solution which comprises the G2/M cycle inhibitor, the plant is further subjected to a magnetic field of at least 700 gauss (e., 1350 Gauss) for 20 min to 5 hours. The seeds are placed in a magnetic field chamber such as that described in Example 1. After the indicated time, the seeds are removed from the magnetic field.

To improve permeability of the seeds to the treatment solution, the seeds are subjected to ultrasound treatment (e.g., 17-40 kHz 5-40 min) prior to contacting with the G2/M cycle inhibitor.

Wet seeds may respond better to treatment and therefore seeds are soaked in an aqueous solution (e.g., distilled water) at the initiation of treatment.

According to a specific embodiment, the entire treatment is performed in the dark and at room temperature (about 23-26° C.) or lower (e.g., for the US stage).

Thus according to a specific embodiment, the seeds are soaked in water at room temperature and then subjected to US treatment in distilled water.

Once permeated, the seeds are placed in a receptacle containing the treatment solution and a magnetic field in turned on. Exemplary ranges of G2/M cycle inhibitor concentrations are provided in Table 2 below. The treatment solution may further comprises DMSO, detergents, antioxidants and histones at the concentrations listed below.

Once the seeds removed from the magnetic field they are subject to a second round of treatment with the G2/M cycle inhibitor. Finally, the seeds are washed and seeded on appropriate growth beds. Optionally, the seedlings are grown in the presence of Acadain™ (Acadian AgriTech) and Giberllon (the latter is used when treated with vinblastine, as the G2/M cycle inhibitor).

It will be appreciated that the above method may be implemented on the whole plant or plant part such as described herein and not necessarily restricted to seeds.

Using the above teachings, the present inventors have established genomically multiplied common wheat plants.

Once established, the plants of the present invention can be propagated sexually or asexually such as by using tissue culturing techniques.

As used herein the phrase “tissue culture” refers to plant cells or plant parts from which wheat grass can be generated, including plant protoplasts, plant cali, plant clumps, and plant cells that are intact in plants, or part of plants, such as seeds, leaves, stems, pollens, roots, root tips, anthers, ovules, petals, flowers, embryos, grains, fibers and bolls. Of note, the terms seeds and grains are interchangeably used herein.

According to some embodiments of the present invention, the cultured cells exhibit genomic stability for at least 2, 3, 4, 5, 7, 9 or 10 passages in culture.

Techniques of generating plant tissue culture and regenerating plants from tissue culture are well known in the art. For example, such techniques are set forth by Vasil., 1984. Cell Culture and Somatic Cell Genetics of Plants, Vol I, II, III, Laboratory Procedures and Their Applications, Academic Press, New York; Green et al., 1987. Plant Tissue and Cell Culture, Academic Press, New York; Weissbach and Weissbach. 1989. Methods for Plant Molecular Biology, Academic Press; Gelvin et al., 1990, Plant Molecular Biology Manual, Kluwer Academic Publishers; Evans et al., 1983, Handbook of Plant Cell Culture, MacMillian Publishing Company, New York; and Klee et al., 1987. Ann. Rev. of Plant Phys. 38:467 486.

The tissue culture can be generated from cells or protoplasts of a tissue selected from the group consisting of seeds, leaves, stems, pollens, roots, root tips, anthers, ovules, petals, flowers and embryos.

It will be appreciated that the plants of the present invention can also be used in plant breeding along with other wheat plants (i.e., self-breeding or cross breeding) in order to generate novel plants or plant lines which exhibit at least some of the characteristics of the common wheat plants of the present invention.

Plants resultant from crossing any of these with another plant can be utilized in pedigree breeding, transformation and/or backcrossing to generate additional cultivars which exhibit the characteristics of the genomically multiplied plants of the present invention and any other desired traits. Screening techniques employing molecular or biochemical procedures well known in the art can be used to ensure that the important commercial characteristics sought after are preserved in each breeding generation.

The goal of backcrossing is to alter or substitute a single trait or characteristic in a recurrent parental line. To accomplish this, a single gene of the recurrent parental line is substituted or supplemented with the desired gene from the nonrecurrent line, while retaining essentially all of the rest of the desired genes, and therefore the desired physiological and morphological constitution of the original line. The choice of the particular nonrecurrent parent will depend on the purpose of the backcross. One of the major purposes is to add some commercially desirable, agronomically important trait to the plant. The exact backcrossing protocol will depend on the characteristic or trait being altered or added to determine an appropriate testing protocol. Although backcrossing methods are simplified when the characteristic being transferred is a dominant allele, a recessive allele may also be transferred. In this instance, it may be necessary to introduce a test of the progeny to determine if the desired characteristic has been successfully transferred. Likewise, transgenes can be introduced into the plant using any of a variety of established transformation methods well-known to persons skilled in the art, such as: Gressel., 1985. Biotechnologically Conferring Herbicide Resistance in Crops: The Present Realities, In: Molecular Form and Function of the plant Genome, L van Vloten-Doting, (ed.), Plenum Press, New York; Huftner, S. L., et al., 1992, Revising Oversight of Genetically Modified Plants, Bio/Technology; Klee, H., et al., 1989, Plant Gene Vectors and Genetic Transformation: Plant Transformation Systems Based on the use of Agrobacterium tumefaciens, Cell Culture and Somatic Cell Genetics of Plants; and Koncz, C., et al. 1986, Molecular and General Genetics.

It will be appreciated that plants or hybrid plants of the present invention can be genetically modified such as in order to introduce traits of interest e.g. enhanced resistance to stress (e.g., biotic or abiotic).

Thus, the present invention provides novel genomically multiplied plants and cultivars, and seeds and tissue culture for generating same.

The plant of the present invention is capable of self-breeding or cross-breeding with a hexaploid or tetraploid wheat or wheat of various ploidies (e.g., induced high-ploidy wheat as described herein) or with other wheat species.

Thus, the present invention further provides for a hybrid plant having as a parental ancestor the genomically multiplied plant as described herein.

For instance, the male parent may be the genomically multiplied plant while the female parent may be a hexaploid common wheat or even a tetraploid Durum wheat plant (interspecies crossing).

According to a specific embodiment the invention provides for a hybrid common wheat plant having a partially or fully multiplied genome.

The present invention further provides for a seed bag which comprises at least 10%, 20% 50% or 100% (say 10-100%) of the seeds of the plants or hybrid plants of the invention.

Using the present teachings, the present inventors were able to generate a number of plant varieties which are induced polyploids. A sample of representative seeds of a common wheat plant having a partially or fully multiplied genome being at least as fertile as a hexaploid common wheat (Triticum aestivum L.) plant isogenic to said genomically multiplied common wheat plant when grown under the same conditions, wherein said sample has been deposited under the Budapest Treaty at the NCIMB under NCIMB 41972 on May 18, 2012.

The present invention further provides for a planted field which comprises any of the plants or hybrid plants of the invention.

Grains of the present invention are processed as meal used as supplements in leavened, flat and steamed breads, biscuits, cookies, cakes, pastries, snacks, breakfast cereal, pasta, noodles and couscous.

Accordingly, the present invention further provides for a method of producing common wheat meal, the method comprising harvesting grains of the plant or hybrid plant of the invention; and processing the grains so as to produce meal.

Wheat grass is highly fermentable, which makes the plants or hybrids of the invention a good alternative for use in beer and other alcoholic beverages production and also useful for production of biofuels. Plants or hybrids of the invention can also be used in construction (e.g., straw), such as a thatch for roofing.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Generation of Genomically Multiplied Common Wheat

All steps are performed in the dark.
Soak seeds in a vessel full of water at 25° C. for about 2 hr.
Transfer the seeds into a clean net bag.
Insert into a distilled water filled ultrasonic bath at 23-26° C.
Apply sonication (40 KHz) for 5 to 20 minutes. Keep temperature below 26° C.
Place seeds bag into a vessel containing the treatment solution at about 25° C.
Place the vessel within the magnetic field chamber (as described in details below) and incubate for about 2 hr.
Remove seeds from the bag and place on top of a paper towels bed on a plastic tray.
Cover with a second layer of paper towel and soak with treatment solution.
Incubate for 12-48 hr at about 25° C. Keep wet for the whole incubation period.
Collect the seeds into a clean vessel and wash with water (pH=7).
Prepare a seedling tray of soil supplemented with 25 ppm of 20:20:20 Micro Elements Fertilizer.
Seed treated seeds to the tray and move to nursery using a day temperature range of 20-25° C., night range of 10-17° C. and minimal moisture of 40%.
When using Vinblastine, treat with 0.5-1.5% GIBERLLON immediately after seeding.
Treat with ACADIAN™ (Acadian, AgriTech) twice a week for the following 3 weeks.

### Treatment Solution:

### DMSO 0.5%

TritonX100 5 drops/L
microtubule polymerization inhibitor

### Antioxidant

Histones 50-100 ug/ml
pH=6
Prepare in softened, nitrogen free water
Use immediately.

TABLE 1

| Concentration | Microtubule polymerization inhibitor |
|---|---|
| 0.05-0.2% | Vinblastine sulphate |
| 0.1-0.5 mg/ml | Colchicine |
| 0.1-0.9% | Nocodazole |
| 0.002-0.005% | Oryzalin |
| 0.002-0.005% | Trifluralin |

TABLE 2

| Concentration | Antioxidants |
|---|---|
| 25-100 ug/ml | Cyanidin 3-O-b-glucopyranoside |
| 10⁻⁶⁻10⁻⁴M | Baicalein |
| 10⁻⁶⁻10⁻⁴M | Quercetin |
| 5-10 mM | Trolox |

### Magnetic Field Chamber for Treatments

The magnetic field chamber consisted of two magnet boards located 11 cm from each other.

The magnetic field created by the two magnets is a coil-shaped magnetic field with a minimal strength of 1350 gauss in the central solution.

Magnetic Field Chamber

The magnetic field chamber consisted of two magnet boards located 11 cm from each other. The magnetic field formed by the two magnets is a coil-shaped magnetic field with a minimal strength of 1350 gauss in its treatment solution treatment solution at pH=6 which includes 0.5% DMSO, 5 drops/L TritonX100, microtubule polymerization inhibitor, antioxidant’ 100 ug/ml Histones at 22° C. The bath was inserted into the magnetic chamber.

The seeds were placed in a net bag within a stainless steel bath filled with treatment solution, and the bath was inserted into the magnetic chamber.

### Example 2

### General Materials and Methods for Phenotypic Characterization

Ploidy Examination Using Flow Cytometry

Samples of nuclei for Flow cytometry were prepared from leaves. Each sample (1 cm²) was chopped with a razor blade in a chopping buffer consisting of, 9.15 g MgCl₂, 8.8 g sodium citrate, 4.19 g 3-[morpholino]propane sulfonic acid, 1 ml Triton X-100, 21.8 g sorbitol per liter. The resulting slurry was filtered through a 23 μm nylon mesh, and Propidium Iodide (PI) was added to a final concentration of 0.2 mg/L. The stained samples were stored on ice and analyzed by flow cytometry. The flow cytometer was a FACSCalibur (BD Biosciences ltd.).

Breeder Selection

Genome multiplication protocol (see Genome Multiplication Procedure under Material and Methods section), applied on various male control lines. Plants were selected for high ploidy according to their phenotype in the field. The phenotypic analyses included a number of parameters including leaf color, leaf thickness, seed color, seed size. Thereafter, FACS analysis (as described above) confirmed that the plants and their offspring were of stable (D1 and D2) high ploidy. The plants were self-crossed as follows; covering the inflorescence of the female before the spiklets had matured (before the stigmas were receptive). Covering of the inflorescence ensures that the pollination is a self-pollination. The flowers were hermaphrodites (both male and female). Pollination occurred spontaneously. Seeds were harvested upon maturity.

Following basic field preparation-all plots were seeded with commercial common wheat as well as the experimental varieties. First, seeds were seeded in the nursery and then transplanted to rows (for single plants) or plots (0.5 m², 10 plants). Single plants trial program, was transplanted in rows, with an average of 2-10 plants. In addition, polyploid hybrids trail program was performed in 0.5×1 m trial plots with 10 plants on each plot in 1 replica.

### Example 3

### Phenotypic Characterization of Genomically Multiplied Bread Wheat

The multiplied wheat was generated according to the protocol provided in Example 1, above. Five wheat lines from different backgrounds were treated. Selected plants that went through the process successfully were evaluated by FACS (analyzing nuclei population according to DNA content). The offspring was sown and phenotype was checked again by FACS. The process was repeated on 5 additional lines (results not shown).

In addition to that, a few combinations between these 5 lines and different lines possessing potential heterosis were tested.

Altogether 18 families of three different backgrounds (each family derived from different successfully treated plant) that show stable high ploidy level were obtained: 8 of R-2010-1, 2 of BB-2010-2 and 8 of SM-2010-3. These plants are second generation after treatment (“D2”). See Table 3.

TABLE 3
High ploidy families of Wheat lines. Each plant family are the
self-seeds of different successfully genome multiplied plant.
D2 indicates that the plants are second
generation after treatment.
2010-1 of Table 5 is 336
2010-4 of Table 5 is 128

| | Control | FACS | | | |
|---|---|---|---|---|---|
| Comments | result | result | Generation | Name | Background |
| Addition of 1 set | 280-300 | 350 | D2 | 206 | SM-2010-3 |
| Addition of 1 set | 280-300 | 370 | D2 | 221 | |
| Addition of 1 set | 280-300 | 380 | D2 | 245 | |
| Addition of 1 set | 280-300 | 360 | D2 | 254 | |
| Addition of 1 set | 280-300 | 360 | D2 | 257 | |
| Addition of 1 set | 280-300 | 360 | D2 | 274 | |
| Addition of 1 set | 280-300 | 400 | D2 | 316 | |
| Addition of 1 set | 280-300 | 350 | D2 | 321 | |
| Addition of 1 set | 280-300 | 380 | D2 | 336 | R-2010-1 |
| Addition of 1 set | 280-300 | 370 | D2 | 368 | |
| Addition of 1 set | 280-300 | 370 | D2 | 375 | |
| Addition of 1 set | 280-300 | 360 | D2 | 389 | |
| Addition of 1 set | 280-300 | 380 | D2 | 390 | |
| Addition of 1 set | 280-300 | 360 | D2 | 391 | |
| Addition of 1 set | 280-300 | 360 | D2 | 394 | |
| Addition of | 300 | 620 | D2 | 460 | BB-2010-2 |
| all 3 sets | | | | | |
| Addition of 2 sets | 300 | 460 | D2 | 468 | |
| Addition of | 300 | 620 | D2 | 470 | |
| all 3 sets | | | | | |

42 new polyploid plants of all 5 backgrounds were achieved: 15 of R 2010-1, 9 of BB-2010-2, 5 of SM-2010-3, 21 of H-2010-4 and 13 of C0-2010-5. These are the treated plants (“D1”). See Table 4, below.

TABLE 4
High ploidy plants Wheat lines. Each row\number presents a different
successfully genome multiplied plant. D1 indicates that these are the
treated plants.

| | Control | FACS | Genera- | | |
|---|---|---|---|---|---|
| Comments | result | result | tion | Name | Background |
| Addition of 1 set | 280 | 380 | D1 | 2 | R-2010-1 |
| Addition of 1 set | 280 | 360 | D1 | 3 | |
| Addition of 1 set | 280 | 360 | D1 | 4 | |
| Addition of 1 set | 280 | 360 | D1 | 7 | |
| Addition of 1 set | 280 | 360 | D1 | 17 | |
| Addition of 1 set | 280 | 360 | D1 | 22 | |
| Addition of 1 set | 280 | 400 | D1 | 27 | |
| Addition of 1 set | 280 | 400 | D1 | 33 | |
| Addition of 1 set | 280 | 380 | D1 | 39 | |
| Addition of 2 or 3 sets | 280 | 500 | D1 | 45 | |
| Addition of 2 sets | 280 | 440 | D1 | 49 | |
| Addition of 2 or 3 sets | 280 | 500 | D1 | 54 | |
| Addition of 1 set | 280 | 360 | D1 | 68 | |
| Addition of 2 sets | 280 | 440 | D1 | 71 | |
| Addition of 1 set | 280 | 400 | D1 | 122 | |
| Addition of 1 set | 300 | 380 | D1 | 26 | C0-2010-5 |
| Addition of all 3 sets | 300 | 600 | D1 | 34 | |
| Addition of all 3 sets | 300 | 600 | D1 | 36 | |
| Addition of 2 sets | 300 | 400 | D1 | 41 | |
| Addition of 2 sets | 300 | 400 | D1 | 43 | |
| Addition of 2 or 3 sets | 300 | 540 | D1 | 50 | |
| Addition of 2 sets | 300 | 400 | D1 | 59 | |
| Addition of 2 sets | 300 | 400 | D1 | 63 | |
| Addition of 2 sets | 300 | 400 | D1 | 54 | |
| Addition of 2 or 3 sets | 300 | 500 | D1 | 71 | |
| Addition of 2 sets | 300 | 400 | D1 | 103 | |
| Addition of 2 sets | 300 | 460 | D1 | 104 | |
| Addition of 2 or 3 sets | 300 | 500 | D1 | 132 | |
| Addition of 2 sets | 280 | 460 | D1 | 6 | BB-2010-2 |
| Addition of 2 or 3 sets | 280 | 500 | D1 | 14 | |
| Addition of all 3 sets | 280 | 600 | D1 | 40 | |
| Addition of 1 set | 280 | 380 | D1 | 42 | |
| Addition of 2 or 3 sets | 280 | 560 | D1 | 43 | |
| Addition of all 3 sets | 280 | 600 | D1 | 55 | |
| Addition of all 3 sets | 280 | 600 | D1 | 94 | |
| xxx | 280 | 340 | D1 | 117 | |
| Addition of 2 or 3 sets | 280 | 500 | D1 | 134 | |
| Addition of 1 set | 280 | 370 | D1 | 36 | SM-2010-3 |
| Addition of 1 set | 280 | 370 | D1 | 64 | |
| Addition of 1 set | 280 | 380 | D1 | 94 | |
| Addition of 1 set | 280 | 360 | D1 | 106 | |
| Addition of 1 set | 280 | 380 | D1 | 123 | |
| Addition of 1 or 2 sets | 300 | 440 | D1 | 5 | H-2010-4 |
| Addition of all 3 sets | 300 | 700 | D1 | 6 | |
| Addition of all 3 sets | 300 | 600-800 | D1 | 13 | |
| Addition of 1 set | 300 | 400 | D1 | 17 | |
| Addition of 2 or 3 sets | 300 | 520 | D1 | 21 | |
| Addition of all 3 sets | 300 | 800 | D1 | 23 | |
| addition of 1 or 2 sets | 300 | 450 | D1 | 24 | |
| addition of 1 or 2 sets | 300 | 420 | D1 | 40 | |
| Addition of 2 or 3 sets | 300 | 540 | D1 | 41 | |
| Addition of 1 set | 300 | 400 | D1 | 68 | |
| Addition of 2 or 3 sets | 300 | 460 | D1 | 70 | |
| Addition of 1 set | 300 | 400 | D1 | 72 | |
| Addition of all 3 sets | 300 | 600 | D1 | 80 | |
| Addition of 1 set | 300 | 400 | D1 | 83 | |
| Addition of 2 or 3 sets | 300 | 520 | D1 | 88 | |
| Addition of 1 set | 300 | 380 | D1 | 96 | |
| addition of 2 sets | 300 | 480 | D1 | 103 | |
| Addition of 1 set | 300 | 400 | D1 | 114 | |
| addition of 2 sets | 300 | 500 | D1 | 121 | |
| Addition of 2 or 3 sets | 300 | 520 | D1 | 128 | |
| Addition of all 3 sets | 300 | 620 | D1 | 129 | |

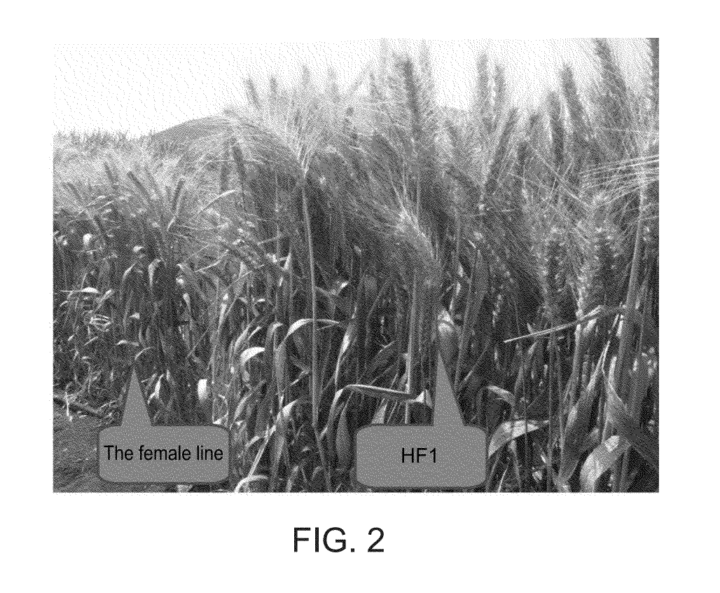

In addition, a few combinations that show strong heterosis between normal ploidy lines (female) and 5 high ploidy lines (male) were obtained. Results are shown in FIGS. 2 and 3.

Tables 5-11 and on substantiate the superiority of the geneomically multiplied males and their potential use in future hybrid production.

TABLE 5

| tolerance | yield/ | | total | grain/ | | | spike | plant | |
|---|---|---|---|---|---|---|---|---|---|
| to rust | plant 10 gr | weight/1000 | grain/plant/100 | spikelet | spikelet | spikes | length | length | Codes |
| 1-sus′-5 resist′ | 10 grams | grams | # | # | # | # | cm | cm | Units |
| 4 | 34.3728 | 42 | 81.84 | 4 | 33 | 62 | 13 | 80 | HF1code: W2(620) |
| 2 | 14.3964 | 43 | 33.48 | 4 | 27 | 31 | 10 | 70 | Female line: 3-2010(30)2 |
| 4 | 16.2 | 40 | 40.5 | 3 | 30 | 45 | 12 | 80 | Male line: 11-2010(308)1 |
| 4 | 20.304 | 45 | 45.12 | 3 | 32 | 47 | 14 | 70 | 12 N isogenic male line: |
| 3 | 33.0132 | 41 | 80.52 | 4 | 33 | 61 | 12 | 80 | HF1code: W15(659) |
| 2 | 17.472 | 40 | 43.68 | 4 | 26 | 42 | 8 | 70 | Female line: 8-2010(520)1 |
| 2 | 12.3 | 41 | 30 | 4 | 25 | 30 | 10 | 70 | Male line: 3-2010(30)2 |
| 3 | 15.7696 | 44 | 35.84 | 4 | 28 | 32 | 12 | 80 | 12 N isogenic male line: |
| 3 | 31.668 | 42 | 75.4 | 5 | 29 | 52 | 11 | 90 | HF1code: W16(648) |
| 2 | 18.8272 | 41 | 45.92 | 4 | 28 | 41 | 8 | 70 | Female line: 7-2010(130)1 |
| 3 | 14.58 | 40 | 36.45 | 3 | 27 | 45 | 10 | 90 | Male line: R-2010-1 |
| 3 | 18.576 | 43 | 43.2 | 3 | 30 | 48 | 12 | 80 | 8 N isogenic male line: |
| 3 | 24.9312 | 42 | 59.36 | 4 | 28 | 53 | 11 | 80 | HF1code: W17(650) |
| 2 | 18.5976 | 41 | 45.36 | 4 | 27 | 42 | 8 | 70 | Female line: 7-2010(130)1 |
| 3 | 14.04 | 40 | 35.1 | 3 | 26 | 45 | 9 | 70 | Male line: H-2010-4 |
| 3 | 17.9916 | 44 | 40.89 | 3 | 29 | 47 | 11 | 80 | 10 N isogenic male line: |
| 3 | 25.3344 | 42 | 60.32 | 4 | 29 | 52 | 11 | 80 | HF1code: W18(669) |
| 3 | 18.576 | 40 | 46.44 | 4 | 27 | 43 | 8 | 70 | Female line: 8-2010(520)1 |
| 2 | 14.04 | 40 | 35.1 | 3 | 26 | 45 | 10 | 70 | Male line: 13-2010(458)1 |
| 3 | 17.3712 | 44 | 39.48 | 3 | 28 | 47 | 12 | 80 | 12 N isogenic male line: |
| 3 | 24.4608 | 42 | 58.24 | 4 | 28 | 52 | 11 | 80 | HF1code: W19(681) |
| 3 | 18.5976 | 42 | 44.28 | 4 | 27 | 41 | 9 | 70 | Female line: 10-2010(142)4 |
| 2 | 18.72 | 40 | 46.8 | 4 | 26 | 45 | 10 | 70 | Male line: 3-2010(30)2 |
| 3 | 22.4112 | 42 | 53.36 | 4 | 29 | 46 | 12 | 80 | 12 N isogenic male line: |

### HF1 W2 (620)

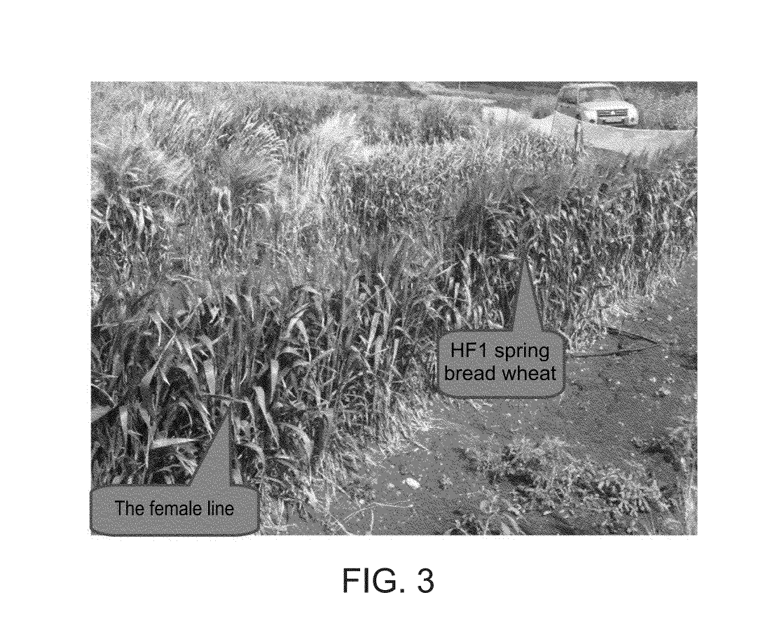

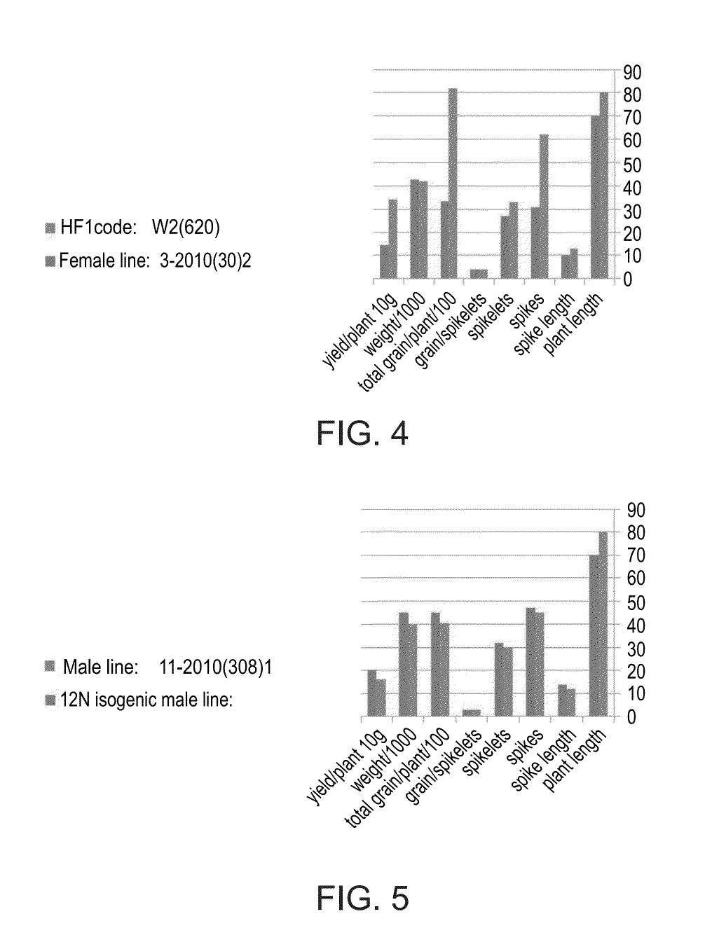

This hybrid is a spring wheat hybrid possessing an excellent heterosis—more than double when compared to the female line. Comparison between the parental male line and the 12N isogenic line shows a large difference in the main parameter—yield per plant.
(See the data below in Table 6 below and FIGS. 4-5).

TABLE 6

| tolerance to | | | total | | | | | | |
|---|---|---|---|---|---|---|---|---|---|
| rust | yield/plant | weight/1000 | grain/plant | grain/spikelet | spikelet | spikes | spike length | plant length | Codes |
| 1-sus′-5 resist′ | 10 gr | grams | #/100 | # | # | # | cm | cm | Units |
| 4 | 34.3728 | 42 | 81.84 | 4 | 33 | 62 | 13 | 80 | HF1code: |
| | | | | | | | | | W2(620) |
| 2 | 14.3964 | 43 | 33.48 | 4 | 27 | 31 | 10 | 70 | Female line: 3- |
| | | | | | | | | | 2010(30)2 |
| 4 | 16.2 | 40 | 40.5 | 3 | 30 | 45 | 12 | 80 | Male line: 11- |
| | | | | | | | | | 2010(308)1 |
| 4 | 20.304 | 45 | 45.12 | 3 | 32 | 47 | 14 | 70 | 12 N isogenic |
| | | | | | | | | | male line: |

| | | total | | | | | | |
|---|---|---|---|---|---|---|---|---|
| yield/plant 10 g | weight/1000 | grain/plant/100 | grain/spikelet | spikelet | spikes | spike length | plant length | |
| 34.3728 | 42 | 81.84 | 4 | 33 | 62 | 13 | 80 | HF1code: |
| | | | | | | | | W2(620) |
| 14.3964 | 43 | 33.48 | 4 | 27 | 31 | 10 | 70 | Female line: 3- |
| | | | | | | | | 2010(30)2 |
| 16.2 | 40 | 40.50 | 3 | 30 | 45 | 12 | 80 | Male line: 11- |
| | | | | | | | | 2010(308)1 |
| 20.304 | 45 | 45.12 | 3 | 32 | 47 | 14 | 70 | 12 N isogenic male |
| | | | | | | | | line: |

### HF1 W 15 (659):

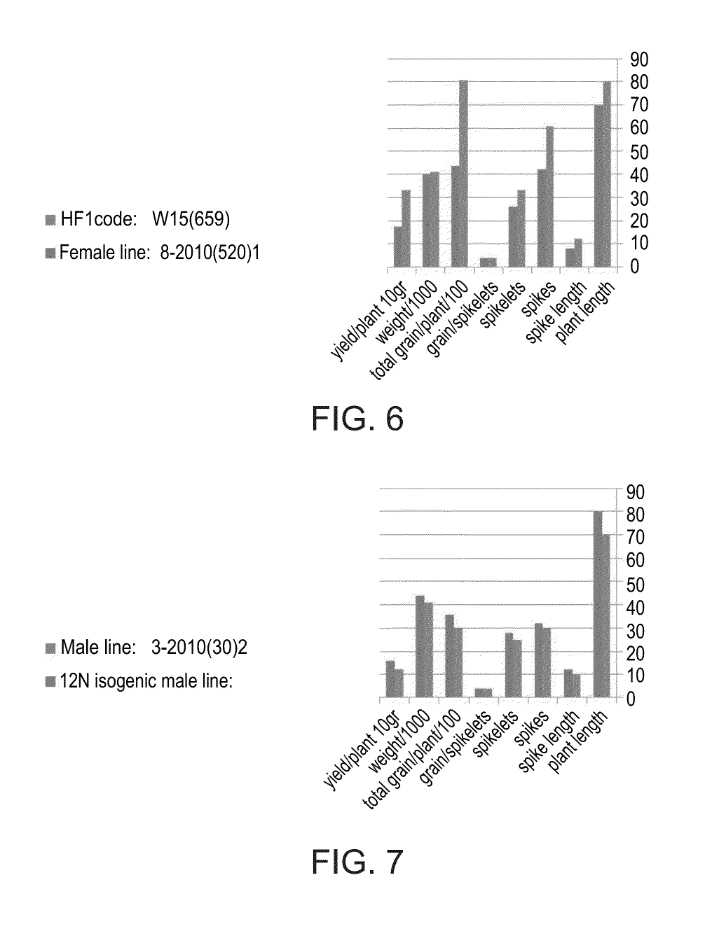

This is a spring wheat variety demonstrating extremely high heterosis when compared to the female line.
Comparison between the male line and the isogenic 12N line demonstrate large difference in all the main parameters (see the data below in Table 7 and FIGS. 6-7).

TABLE 7

| tolerance to | yield/plant | | total | | | | | plant | |
|---|---|---|---|---|---|---|---|---|---|
| rust | 100 gr | weight/1000 | grain/plant/100 | grain/spikelet | spikelet | spikes | spike length | length | Codes |
| 1-sus′-5 resist′ | 100 grams | grams | #/100 | # | # | # | cm | cm | Units |
| 3 | 33.0132 | 41 | 80.52 | 4 | 33 | 61 | 12 | 80 | HF1code: |
| | | | | | | | | | W15(659) |
| 2 | 17.472 | 40 | 43.68 | 4 | 26 | 42 | 8 | 70 | Female line: 8- |
| | | | | | | | | | 2010(520)1 |
| 2 | 12.3 | 41 | 30 | 4 | 25 | 30 | 10 | 70 | Male line: 3- |
| | | | | | | | | | 2010(30)2 |
| 3 | 15.7696 | 44 | 35.84 | 4 | 28 | 32 | 12 | 80 | 12 N isogenic |
| | | | | | | | | | male line: |

| | | total | | | | | | |
|---|---|---|---|---|---|---|---|---|
| yield/plant 10 gr | weight/1000 | grain/plant/100 | grain/spikelet | spikelet | spikes | spike length | plant length | |
| 33.0132 | 41 | 80.52 | 4 | 33 | 61 | 12 | 80 | HF1code: |
| | | | | | | | | W15(659) |
| 17.472 | 40 | 43.68 | 4 | 26 | 42 | 8 | 70 | Female line: 8- |
| | | | | | | | | 2010(520)1 |
| 12.3 | 41 | 30 | 4 | 25 | 30 | 10 | 70 | Male line: 3- |
| | | | | | | | | 2010(30)2 |
| 15.7696 | 44 | 35.84 | 4 | 28 | 32 | 12 | 80 | 12 N isogenic male |
| | | | | | | | | line: |

### HF1 W 16(648)

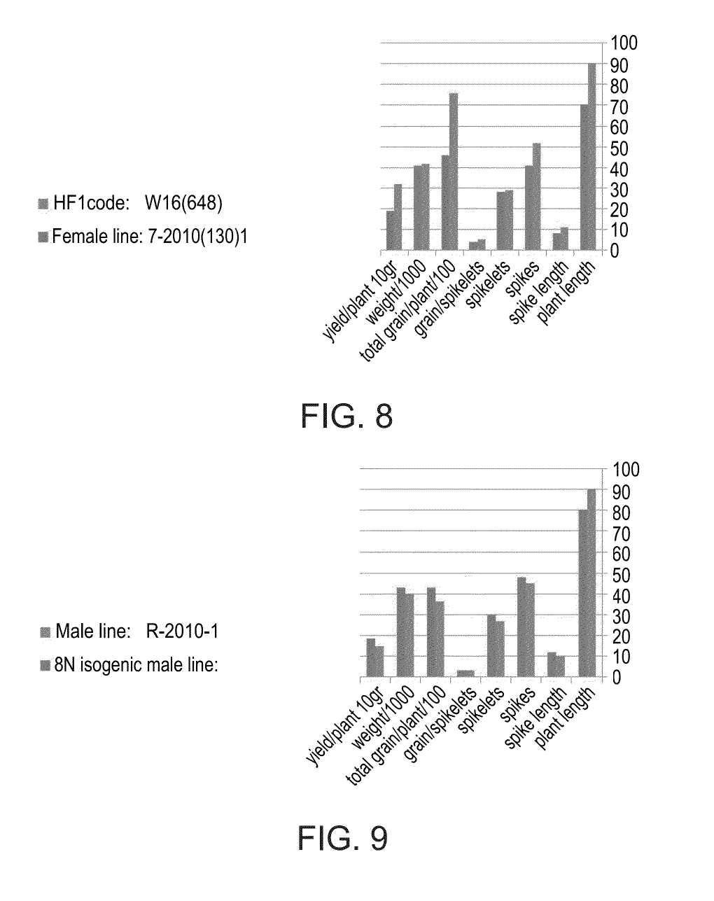

This is a winter wheat variety demonstrating extremely high heterosis when compared to the female line.
Comparison between the male line and the isogenic 8N line demonstrate large difference in all the main parameters (see the data below in Table 8 and FIGS. 8-9).

TABLE 8

| tolerance to | | | total | | | | | | |
|---|---|---|---|---|---|---|---|---|---|
| rust | yield/plant 10 gr | weight/1000 | grain/plant/100 | grain/spikelet | spikelet | spikes | spike length | plant length | Codes |
| 1-sus′-5 resist′ | 10 grams | grams | # | # | # | # | cm | cm | Units |
| 3 | 31.668 | 42 | 75.4 | 5 | 29 | 52 | 11 | 90 | HF1code: |
| | | | | | | | | | W16(648) |
| 2 | 18.8272 | 41 | 45.92 | 4 | 28 | 41 | 8 | 70 | Female line: 7- |
| | | | | | | | | | 2010(130)1 |
| 3 | 14.58 | 40 | 36.45 | 3 | 27 | 45 | 10 | 90 | Male line: R- |
| | | | | | | | | | 2010-1 |
| 3 | 18.576 | 43 | 43.2 | 3 | 30 | 48 | 12 | 80 | 8 N isogenic male |
| | | | | | | | | | line: |

| | | total | | | | | | |
|---|---|---|---|---|---|---|---|---|
| yield/plant 10 gr | weight/1000 | grain/plant/100 | grain/spikelet | spikelet | spikes | spike length | plant length | |
| 31.668 | 42 | 75.4 | 5 | 29 | 52 | 11 | 90 | HF1code: |
| | | | | | | | | W16(648) |
| 18.8272 | 41 | 45.92 | 4 | 28 | 41 | 8 | 70 | Female line: 7- |
| | | | | | | | | 2010(130)1 |
| 14.58 | 40 | 36.45 | 3 | 27 | 45 | 10 | 90 | Male line: R- |
| | | | | | | | | 2010-1 |
| 18.576 | 43 | 43.2 | 3 | 30 | 48 | 12 | 80 | 8 N isogenic male |
| | | | | | | | | line: |

### HF1W 17(650):

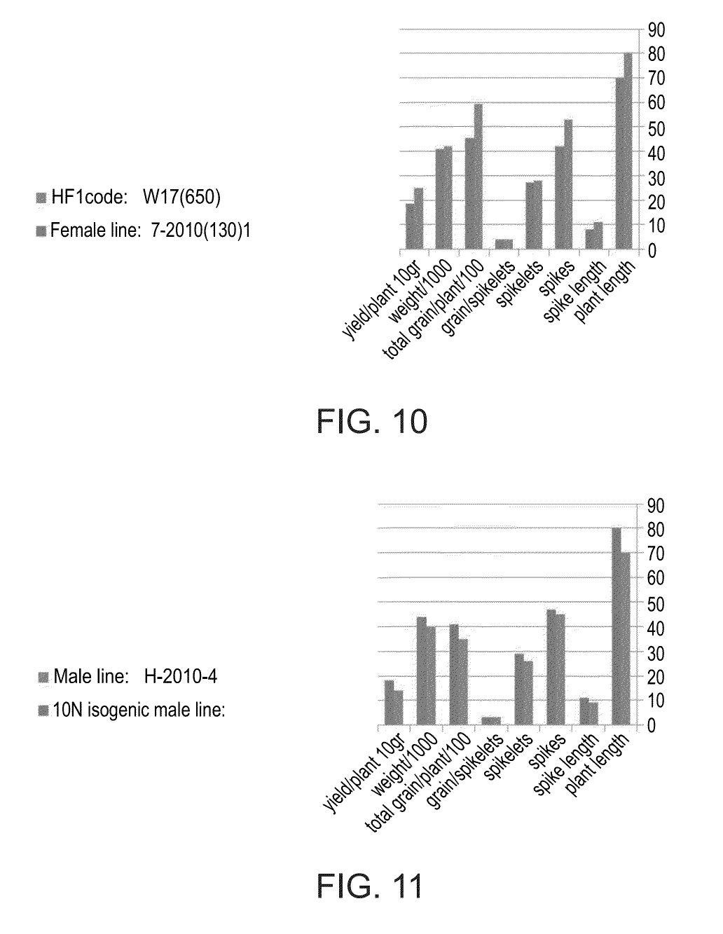

This is a winter wheat variety demonstrating extremely high heterosis when compared to the female line.
Comparison between the male line and the isogenic 10N line demonstrate large difference in all the main parameters (see the data below in Table 9, FIGS. 10-11).

TABLE 9

| tolerance to | | | total | | | | spike | plant | |
|---|---|---|---|---|---|---|---|---|---|
| rust | yield/plant 10 gr | weight/1000 | grain/plant/100 | grain/spikelet | spikelet | spikes | length | length | Codes |
| 1-sus′-5 resist′ | 10 grams | grams | # | # | # | # | cm | cm | Units |
| 3 | 24.9312 | 42 | 59.36 | 4 | 28 | 53 | 11 | 80 | HF1code: |
| | | | | | | | | | W17(650) |
| 2 | 18.5976 | 41 | 45.36 | 4 | 27 | 42 | 8 | 70 | Female line: 7- |
| | | | | | | | | | 2010(130)1 |
| 3 | 14.04 | 40 | 35.1 | 3 | 26 | 45 | 9 | 70 | Male line: H- |
| | | | | | | | | | 2010-4 |
| 3 | 17.9916 | 44 | 40.89 | 3 | 29 | 47 | 11 | 80 | 10 N isogenic |
| | | | | | | | | | male line: |

| | | total | | | | | | |
|---|---|---|---|---|---|---|---|---|
| yield/plant 10 gr | weight/1000 | grain/plant/100 | grain/spikelet | spikelet | spikes | spike length | plant length | |
| 24.9312 | 42 | 59.36 | 4 | 28 | 53 | 11 | 80 | HF1code: |
| | | | | | | | | W17(650) |
| 18.5976 | 41 | 45.36 | 4 | 27 | 42 | 8 | 70 | Female line: 7- |
| | | | | | | | | 2010(130)1 |
| 14.04 | 40 | 35.1 | 3 | 26 | 45 | 9 | 70 | Male line: H- |
| | | | | | | | | 2010-4 |
| 17.9916 | 44 | 40.89 | 3 | 29 | 47 | 11 | 80 | 10 N isogenic |
| | | | | | | | | male line: |

### HF1 W 18(669):

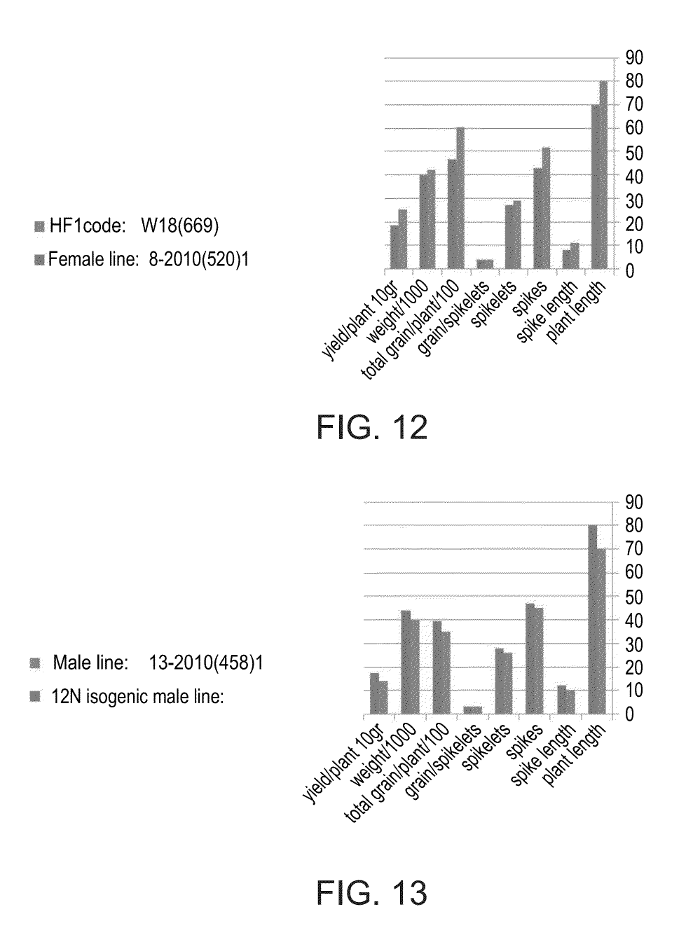

This is a winter wheat variety demonstrating extremely high heterosis when compared to the female line.
Comparison between the male line and the isogenic 12N line demonstrate large difference in all the main parameters (see the data below in Table 10 and FIGS. 12-13).

TABLE 10

| tolerance to | | | total | | | | spike | plant | |
|---|---|---|---|---|---|---|---|---|---|
| rust | yield/plant 10 gr | weight/1000 | grain/plant/100 | grain/spikelet | spikelet | spikes | length | length | Codes |
| 1-sus′-5 resist′ | 10 grams | grams | # | # | # | # | cm | cm | Units |
| 3 | 25.3344 | 42 | 60 | 4 | 29 | 52 | 11 | 80 | HF1code: |
| | | | | | | | | | W18(669) |
| 3 | 18.576 | 40 | 46 | 4 | 27 | 43 | 8 | 70 | Female line: 8- |
| | | | | | | | | | 2010(520)1 |
| 2 | 14.04 | 40 | 35 | 3 | 26 | 45 | 10 | 70 | Male line: 13- |
| | | | | | | | | | 2010(458)1 |
| 3 | 17.3712 | 44 | 39 | 3 | 28 | 47 | 12 | 80 | 12 N isogenic |
| | | | | | | | | | male line: |

| | | total | | | | | | |
|---|---|---|---|---|---|---|---|---|
| yield/plant 10 gr | weight/1000 | grain/plant/100 | grain/spikelet | spikelet | spikes | spike length | plant length | |
| 25.3344 | 42 | 60 | 4 | 29 | 52 | 11 | 80 | HF1code: |
| | | | | | | | | W18(669) |
| 18.576 | 40 | 46 | 4 | 27 | 43 | 8 | 70 | Female line: 8- |
| | | | | | | | | 2010(520)1 |
| 14.04 | 40 | 35 | 3 | 26 | 45 | 10 | 70 | Male line: 13- |
| | | | | | | | | 2010(458)1 |
| 17.3712 | 44 | 39 | 3 | 28 | 47 | 12 | 80 | 12 N isogenic male |
| | | | | | | | | line: |

### HF1 W19 (681):

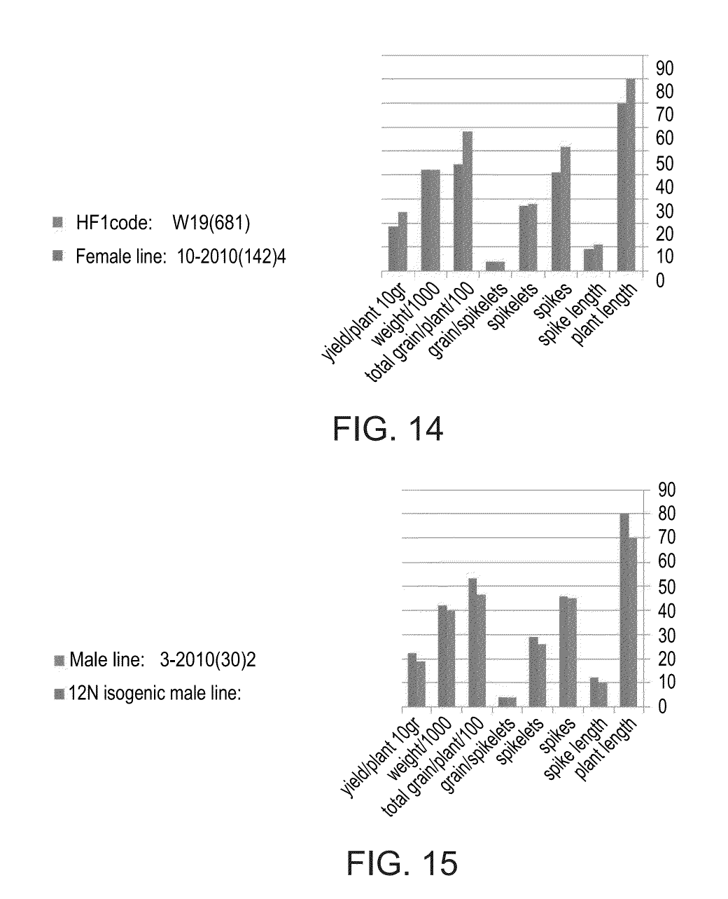

This is a winter wheat variety demonstrating extremely high heterosis when compared to the female line.
Comparison between the male line and the isogenic 12N line demonstrate large difference in all the main parameters (see the data below in Table 11 and FIGS. 14-15).

TABLE 11

| tolerance to | | | total | | | | spike | plant | |
|---|---|---|---|---|---|---|---|---|---|
| rust | yield/plant 10 gr | weight/1000 | grain/plant/100 | grain/spikelet | spikelet | spikes | length | length | Codes |
| 1-sus′-5 resist′ | 10 grams | grams | # | # | # | # | cm | cm | Units |
| 3 | 24.4608 | 42 | 58.2 | 4 | 28 | 52 | 11 | 80 | HF1code: |
| | | | | | | | | | W19(681) |
| 3 | 18.5976 | 42 | 44.3 | 4 | 27 | 41 | 9 | 70 | Female line: |
| | | | | | | | | | 10-2010(142)4 |
| 2 | 18.72 | 40 | 46.8 | 4 | 26 | 45 | 10 | 70 | Male line: 3- |
| | | | | | | | | | 2010(30)2 |
| 3 | 22.4112 | 42 | 53.4 | 4 | 29 | 46 | 12 | 80 | 12 N isogenic |
| | | | | | | | | | male line: |

| | | total | | | | | | |
|---|---|---|---|---|---|---|---|---|
| yield/plant 10 gr | weight/1000 | grain/plant/100 | grain/spikelet | spikelet | spikes | spike length | plant length | |
| 24.4608 | 42 | 58.2 | 4 | 28 | 52 | 11 | 80 | HF1code: |
| | | | | | | | | W19(681) |
| 18.5976 | 42 | 44.3 | 4 | 27 | 41 | 9 | 70 | Female line: 10- |
| | | | | | | | | 2010(142)4 |
| 18.72 | 40 | 46.8 | 4 | 26 | 45 | 10 | 70 | Male line: 3- |
| | | | | | | | | 2010(30)2 |
| 22.4112 | 42 | 53.4 | 4 | 29 | 46 | 12 | 80 | 12 N isogenic male |
| | | | | | | | | line: |

### Example 4

### Phenotypic Characterization of Genomically Multiplied Bread Wheat

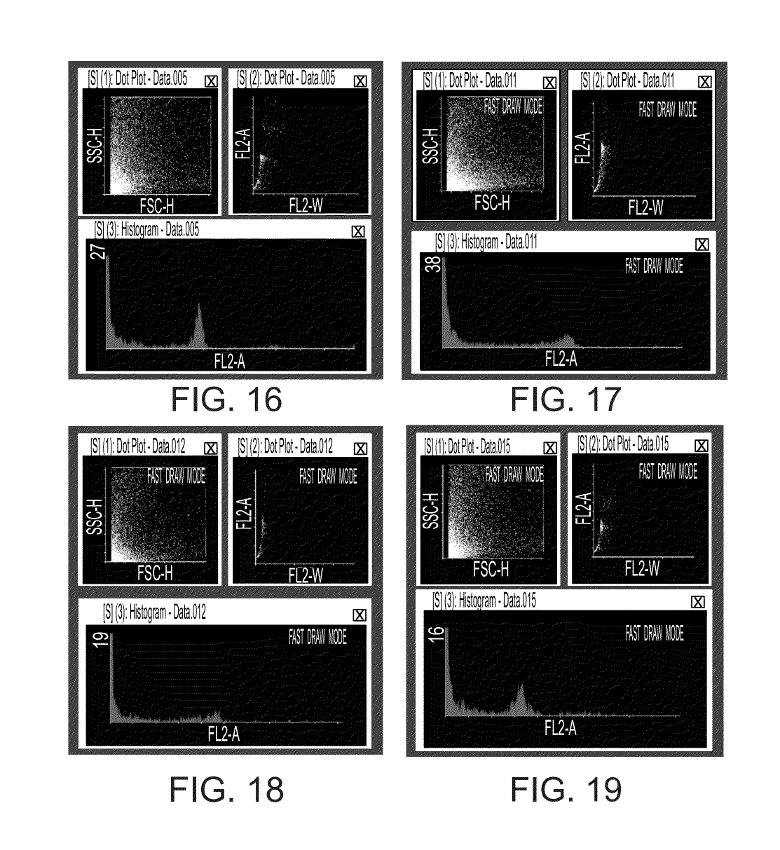

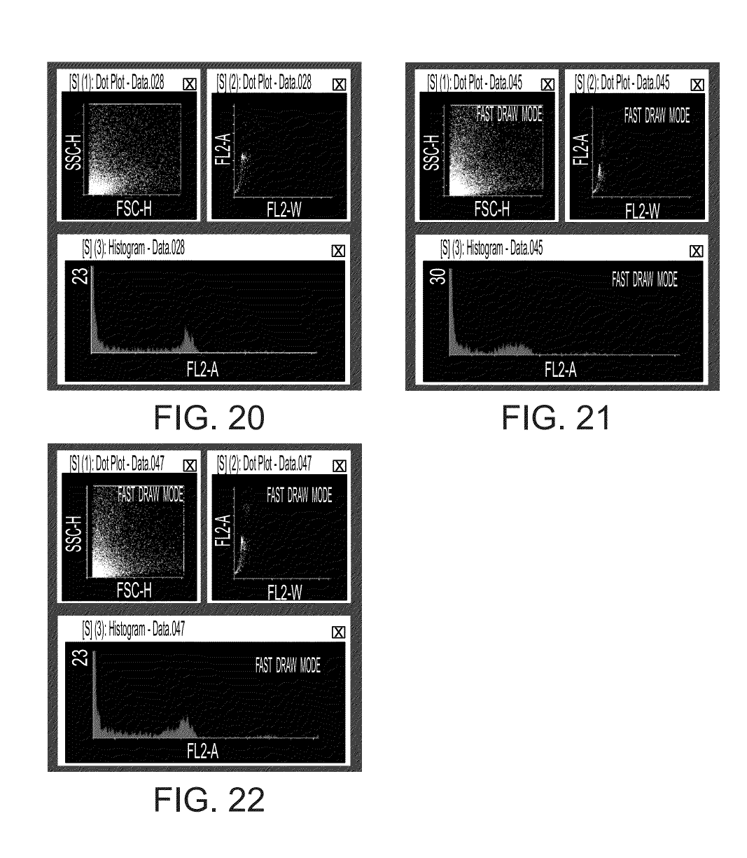

Induced polyploid lines common wheat male plants were generated by subjecting seeds of male hexaploid common wheat to a genome multiplication protocol as described above. The multiplied seeds were referred to as D1. The seeds were placed on a seedling tray containing soil supplemented with fertilizer and moved to a nursery using the above indicated day-night temperature range and minimal moisture as described above. The plants were self-crossed to generate D2 plants of stable induced polyploid lines plants. The genomic stability of polyploid plants was verified by FACS analysis as shown in Table 1, below, and FIGS. 16-22.

Inflorescence was gathered from the plants separately.

The offsprings of D2 was sown and analyzed by FACS analysis. All of the families were confirmed as enhanced polyploid (Table 11 and FIGS. 16-22).

The present results showed that the ploidy of the genomically multiplied common wheat lines was higher than the isogenic line. In addition, it was demonstrated that the induced polyploid lines had more tillers (Table 12, below) as well as larger seeds than the isogenic hexaploid control.

The induced polyploid lines and the hybrids common wheat seeds 32 and 40 found to be larger in shape and size compared to their control isogenic hexaploid line.

Crop yield (total seed weight per plant) of the polyploid plants increased by ten percent compared to control isogenic hexaploid plants (Tables 16, 22 and 29) in both EP (enhanced polyploid or induced polyploid, which are interchangeably used) plants and the polyploid hybrids plant. As common wheat plants are fully self-pollinated and the present results exhibited fertility, all of the above indicate that the EP and polyploid plants had at least equivalent pollen fertility as the control plant.

TABLE 11
Common Wheat Polyploid Lines as Evidenced by FACS Analysis

| | FACS | FACS | | | |
|---|---|---|---|---|---|
| | Results in | Results in | | | |
| | D2 | D1 | Ploidy | | |
| Comments | Generation | Generation | Level | Generation | Name |
| Hexaploid Common Wheat | 320 | 300 | 6n | F6+ | 3-Control |
| Stable High Ploidy Common Wheat | 540 | 480 | EP | D2 | 3-20-1020-1 |
| Stable High Ploidy Common Wheat | 480 | 400 | EP | D2 | 3-20-1030-1 |
| Hexaploid Common Wheat | 320 | 260 | 6n | F6+ | 5-Control |
| Stable High Ploidy Common Wheat | 410 | 420 | EP | D2 | 5-1697-1 |
| Stable High Ploidy Common Wheat | 440 | 400 | EP | D2 | 5-1226-1 |
| Hexaploid Common Wheat | 300 | 280 | 6n | F6+ | 15-control |
| Stable High Ploidy Common Wheat | 420 | 380 | EP | D2 | 15-94 |
| Hexaploid Common Wheat | 220 | 280 | 6n | F6+ | 18-control |
| Stable High Ploidy Common Wheat | 400 | 600 | EP | D1 | 18-55 |

EP—stands for Enhanced polyploid or Induced polyploid lines or induced polyploid.
“3-control”, “5-control”, “15-control” and “18-control” are the isogenic hexaploid lines used for genome multiplication. Each plant family are the self-seeds of different successfully genome multiplied inflorence. D1 and D2 indicates that the plants are first and second generation after genome multiplication procedure respectively. In addition, D1 and D2 represent induced polyploid lines plants whose ploidy is higher than the isogenic source plan, as generated using the protocol of Example 1, above.

TABLE 12
Tiller Number of Induced polyploid lines Compared to Control Lines

| Tillers over | Tiller | Ploidy | | |
|---|---|---|---|---|
| control % | No. | Level | Generation | Name |
| | 28 | 6n | F6+ | 3-control |
| 71.4 | 48 | EP | D2 | 3-20-1020-1 |
| 42.9 | 40 | EP | D2 | 3-20-1030-1 |
| | 24 | 6n | F6+ | 5-control |
| 150 | 60 | EP | D2 | 5-1697-1 |
| 116 | 52 | EP | D2 | 5-1226-1 |

The present results showed that the induced polyploid plants have an increased tiller number at least by 10% compared to the control isogenic line. This data may affect the crop yield.

TABLE 13
Plant Height of Induced polyploid lines Compared to Control Lines

| Plant Height | Ploidy | | |
|---|---|---|---|
| (cm) | Level | Generation | Name |
| 69 | 6n | F6+ | 3-control |
| 78 | EP | D2 | 3-20-1020-1 |
| 77 | EP | D2 | 3-20-1030-1 |
| 80 | 6n | F6+ | 5-control |
| 83 | EP | D2 | 5-1697-1 |
| 86 | EP | D2 | 5-1226-1 |

TABLE 14
Crop Yield of Induced polyploid lines Compared to Control Lines

| Crop Yield Over | Crop Yield | Ploidy | | |
|---|---|---|---|---|
| Control (%) | (Ton/Ha) | Level | Generation | Name |
| | 7.00 | 6n | F6+ | 3-control |
| 229 | 16.00 | EP | D2 | 3-20-1020-1 |
| 247 | 17.30 | EP | D2 | 3-20-1030-1 |
| | 9.30 | 6n | F6+ | 5-control |
| 211 | 19.70 | EP | D2 | 5-1697-1 |
| 203 | 18.90 | EP | D2 | 5-1226-1 |

These findings demonstrate that the induced polyploid plants have at least 2-3 fold higher crop yield as compared to the control isogenic line. Thus, plants exhibited full fertility, indicating at least an equivalent fertility as the control plants.

TABLE 15
Seed Grain Weight of Induced polyploid lines Compared
to Control Lines

| 1000 Seeds Weight | | | | |
|---|---|---|---|---|
| Over Control | 1000 Seeds | Ploidy | | |
| (%) | Weight | Level | Generation | Name |
| | 41.7 | 6n | F6+ | 3-control |
| 116.1 | 48.4 | EP | D2 | 3-20-1020-1 |
| 106.2 | 44.3 | EP | D2 | 3-20-1030-1 |

TABLE 16
Spike Data of Induced polyploid lines Compared to Control Lines

| No. | | | | | | |
|---|---|---|---|---|---|---|
| Seeds | | | | | | |
| per | Spike | Spike | Spike | Ploidy | Gener- | |
| Spikelet | Internodes | Width | Length | Level | ation | Name |
| 5.0 | 13.0 | 2.2 | 14.0 | 6n | F6+ | 3-control |
| 6.0 | 13.0 | 2.5 | 14.5 | EP | D2 | 3-20-1020-1 |
| 6.5 | 13.0 | 2.5 | 16.5 | EP | D2 | 3-20-1030-1 |
| 6.0 | 12.5 | 2.5 | 15.0 | 6n | F6+ | 5-control |
| 5.5 | 13.0 | 2.5 | 14.0 | EP | D2 | 5-1697-1 |
| 6.0 | 13.0 | 2.5 | 13.0 | EP | D2 | 5-1226-1 |

The present results show that the spike length and width were not affected by the genome multiplication protocol in the induced polyploid common wheat plants compared to control. Similarly, the seeds number per spikelet and the spike internodes in the induced-polyploid plants remained the same with no statistically significant differences compared to the isogenic plant.

TABLE 17
Grain Protein Content of Induced polyploid
lines Compared to Control Lines

| Grain Protein | Ploidy | | |
|---|---|---|---|
| Content | Level | Generation | Name |
| 14.0 | 6n | F6+ | 3-control |
| 14.5 | EP | D2 | 3-20-1020-1 |
| 16.5 | EP | D2 | 3-20-1030-1 |
| 15.0 | 6n | F6+ | 5-control |
| 14.0 | EP | D2 | 5-1697-1 |
| 13.0 | EP | D2 | 5-1226-1 |

The present results show that the induced polyploid plants have comparable grain protein which was not affected from the genome multiplication protocol as compared to control.

### Example 5

### Phenotypic Analysis of Polyploid Hybrids

Following basic preparation, the field was sown with common wheat and covered in net. Several spaces shaped as 0.5 m×1.0 m plots were then cleared using herbicide and planted with the experimental plants, including control plants, which were pre-grown in a nursery. Planting density was 10 plants per 0.5 m×1.0 m plot. Each plot contained a single distinct variety or hybrid.

Pre-luminary test of heterosis was made by manual emasculation followed by hand pollination.
“3-control”-Female control (spring wheat), hexaploid.
“5-control”-Female control (spring wheat), hexaploid.
“15-94 D1”-Male EP (winter wheat).
32-polyploid hybrid plant crossed from “3-control” (spring female)×“15-94 D1” (male winter).
40-polyploid hybrid plant crossed from “5-control” (spring female)×“15-94 D1” (male winter).

TABLE 18
Tillers Number of Polyploid Hybrid Plants
Compared to Female Control Lines

| Tillers Over | No. of | Ploidy | | |
|---|---|---|---|---|
| Control (%) | Tillers | Level | Generation | Name |
| | 7 | 6n | F6+ | 3-control |
| 200 | 21 | EP | D1 | 32 polyploid Hybrid |
| | 7 | 6n | F6+ | 5 control |
| 129 | 16 | EP | D1 | 40 polyploid Hybrid |

The common wheat polyploid hybrid has an increased in tiller number in tens to hundreds percentage compared to the female control line under the same developmental stage and growth conditions. This data directly affects the crop yield.

TABLE 19
Plant Height of Polyploid Hybrids Plant Compared to Female Plant

| Plant Height Over | Plant | Ploidy | | |
|---|---|---|---|---|
| Control (%) | Height | Level | Generation | Name |
| | 70 | 6n | F6+ | 3-control |
| 14 | 80 | EP | D1 | 32 polyploid Hybrid |
| | 73 | 6n | F6+ | 5 control |
| 30 | 95 | EP | D1 | 40 polyploid Hybrid |

Thus, the height of hybrid common wheat plant having a partially or fully multiplied genome was differing from the control plant under the same developmental stage and growth conditions. Hence, the crop yield potential is larger in polyploid hybrid plants.

TABLE 20
Seed Grain Weight of Polyploid Hybrids
Plant Compared to Control Plant

| 1000 Seeds | Ploidy | | |
|---|---|---|---|
| Weight | Level | Generation | Name |
| 49.3 | 6n | F6+ | 3-control |
| 49.0 | EP | D1 | 32 polyploid Hybrid |
| 54.0 | 6n | F6+ | 5 control |
| 54.4 | EP | D1 | 40 polyploid Hybrid |

Thus, according to the present results, the polyploid hybrid common wheat plant having a partially or fully multiplied genome were at least as similar in the seeds grain weight compared to the control plant under the same developmental stage and growth conditions.

TABLE 21
Grain Protein Content of Polyploid Hybrids
Plant Compared to Control Plant

| Grain Protein | Ploidy | | |
|---|---|---|---|
| Content | Level | Generation | Name |
| 14.7 | 6n | F6+ | 3-control |
| 13.5 | EP | D1 | 32 polyploid Hybrid |
| 17.6 | 6n | F6+ | 5 control |
| 15.6 | EP | D1 | 40 polyploid Hybrid |

The present results show that the grain protein content was essentially unaffected from the genome multiplication protocol in the polyploid common wheat plants compared to control.

TABLE 22
Crop Yield of Polyploid Hybrids Plant Compared to Control Plant

| Crop Yield | Crop Yield | Ploidy | Gener- | |
|---|---|---|---|---|
| Over Control (%) | (Ton/Ha) | Level | ation | Name |
| | 7.1 | 6n | F6+ | 3-control |
| 49 | 10.4 | EP | D1 | 32 polyploid |
| | | | | Hybrid |
| | 3.0 | 6n | F6+ | 5 control |
| 200 | 9.0 | EP | D1 | 40 polyploid |
| | | | | Hybrid |

The present results show that the polyploid hybrid common wheat plant having a partially or fully multiplied genome has an exceptional increase in the crop yield by up to two hundreds percent compared to the control plant under the same developmental stage and growth conditions. Thus, plants exhibited full fertility indicating that the polyploid hybrid plants had at least equivalent pollen fertility as the control plants.

TABLE 23
Dry Matter Weight of Polyploid Hybrids
Plant Compared to Control Plant

| Dry Matter | Dry Matter | | | |
|---|---|---|---|---|
| Weight per plant | Weight per plant | Ploidy | Gener- | |
| Over Control (%) | (Ton/Ha) | Level | ation | Name |
| | 46.2 | 6n | F6+ | 3-control |
| 105 | 71.8 | EP | D1 | 32 polyploid |
| | | | | Hybrid |
| | 21.3 | 6n | F6+ | 5 control |
| 158 | 55.1 | EP | D1 | 40 polyploid |
| | | | | Hybrid |

Thus the present results show that the polyploid hybrid common wheat plant having a partially or fully multiplied genome demonstrates a significant increase in dry matter weight of over two fold compared to the control plant under the same developmental stage and growth conditions. Hence, the higher quantity of the dry matter weight is indicative of high bio-mass accumulation in the polyploid hybrids plants. In addition, these results indicate that the vigor and the heterosis effect are higher in the hybrid plants compared to control plants.

TABLE 24
Spike Data of Polyploid Hybrids
Compared to Female Control Lines

| No. Seeds | Spike | Spike | | |
|---|---|---|---|---|
| per Spikelet | internodes | width | Spike length | Name |
| 4.5 | 12 | 2.5 | 12.5 | 3-control |
| 3.5 | 12 | 2.0 | 12.0 | 32 polyploid Hybrid |
| 5.0 | 12 | 2.2 | 13.0 | 5 control |
| 4.5 | 13 | 2.2 | 13.5 | 40 polyploid Hybrid |

Thus, the present results show that the spike length and width were not affected by the genome multiplication protocol in the induced hybrid polyploid or from the hybridization crossing in common wheat plants compared to control. Similarly, the seeds number per spikelet and the spike internodes in the hybrids plants remained essentially the same with no statistically significant differences compared to the isogenic plant.

### Example 6

### Phenotypic Characterization of Hybrids Please Find Below

Following basic preparation, the seeds were pre-grown in a nursery and transplanted in rows with inter-row spacing of 25 cm. Data was collected from 3-10 plants.

“3-control”-Female control (spring wheat).
“3-20 D1 EP”-Male EP (spring wheat).
“5-control”-Female control (spring wheat).
“5-79 D1 EP”-Male EP (spring wheat).
“15-94 D1”-Male EP (winter wheat).
“18-control”-Female control (winter wheat).
“18-55 D1 EP”-Male EP (winter wheat).
645-polyploid hybrid plant crossed from “3-control” (spring female)×“15-94 D1 EP” (winter male).
646-polyploid hybrid plant crossed from “3-control” (spring female)×“15-94 D1 EP” (winter male).
649-polyploid hybrid plant crossed from “3-control” (spring female)×“18-55 D1 EP” (winter male).
664-polyploid hybrid plant crossed from “5-control” (spring female)×“15-94 D1 EP” (winter male).
683-polyploid hybrid plant crossed from “18-control” (winter female)×“3-20 D1 EP” (spring male).
685-polyploid hybrid plant crossed from “18-control” (winter female)×“5-79 D1 EP” (winter male).

TABLE 25
Tillers Number of Polyploid Hybrid Plants
Compared to Female Control Lines

| Tillers Over | No. of | Ploidy | | |
|---|---|---|---|---|
| Control (%) | Tillers | Level | Generation | Name |
| | 33 | 6n | F6+ | 3-control |
| | 24 | 6n | F6+ | 5-control |
| | 80 | 6n | F6+ | 18-control |
| 70 | 56 | EP | D1 | 645- polyploid Hybrid |
| 149 | 82 | EP | D1 | 646- polyploid Hybrid |
| 100 | 66 | EP | D1 | 649- polyploid Hybrid |
| 113 | 51 | EP | D1 | 664- polyploid Hybrid |
| 103 | 162 | EP | D1 | 683- polyploid Hybrid |
| ND | ND¹ | EP | D1 | 685- polyploid Hybrid |

¹ND—Not determined

Thus, the common wheat hybrid has an increased in tillers numbers in tens percent compared to the female control line under the same developmental stage and growth conditions. This data directly suggests increased crop yield.

TABLE 26
Plant Height of Polyploid Hybrids Plant Compared to Female Plant

| Plant Height | Plant | Ploidy | | |
|---|---|---|---|---|
| Over Control (%) | Height | Level | Generation | Name |
| | 80 | 6n | F6+ | 3-control |
| | 80 | 6n | F6+ | 5-control |
| | 85 | 6n | F6+ | 18-control |
| 18 | 94 | EP | D1 | 645- polyploid |
| | | | | Hybrid |
| 16 | 93 | EP | D1 | 646- polyploid |
| | | | | Hybrid |
| 16 | 93 | EP | D1 | 649- polyploid |
| | | | | Hybrid |
| 25 | 100 | EP | D1 | 664- polyploid |
| | | | | Hybrid |
| −4 | 82 | EP | D1 | 683- polyploid |
| | | | | Hybrid |
| −6 | 80 | EP | D1 | 685- polyploid |
| | | | | Hybrid |

Thus, the common wheat plant having a partially or fully multiplied genome exhibits essentially the same or higher height than the isogenic hexaploid control. Hence, the crop yield potential is larger in polyploid hybrid plants.

TABLE 27
Seed Grain Weight of Polyploid Hybrids
Plant Compared to Control Plant

| 1000 Seeds | | | | |
|---|---|---|---|---|
| Weight Over | 1000 Seeds | Ploidy | Gener- | |
| Control (%) | Weight | Level | ation | Name |
| | 41.0 | 6n | F6+ | 3-control |
| | 54.3 | 6n | F6+ | 5-control |
| | 28.0 | 6n | F6+ | 18-control |
| 20 | 49.0 | EP | D1 | 645- polyploid Hybrid |
| 23 | 50.5 | EP | D1 | 646- polyploid Hybrid |
| 12 | 46.0 | EP | D1 | 649- polyploid Hybrid |
| ND | ND¹ | EP | D1 | 664- polyploid Hybrid |
| 4 | 29.0 | EP | D1 | 683- polyploid Hybrid |
| 70 | 47.5 | EP | D1 | 685- polyploid Hybrid |

¹ND—Not determined

Thus all the tested grains of the polyploid hybrid common wheat plant having a partially or fully multiplied genome exhibited higher weight compared to the grains of control plant under the same developmental stage and growth conditions. Seeds grain weight is one of the most important yield ingredients. These results support the high crop yield demonstrated in the present analysis.

TABLE 28
Grain Protein Content of Polyploid Hybrids
Plant Compared to Control Plant

| Grain Protein | Ploidy | | |
|---|---|---|---|
| Content | Level | Generation | Name |
| 17.0 | 6n | F6+ | 3-control |
| 17.6 | 6n | F6+ | 5-control |
| 17.6 | 6n | F6+ | 18-control |
| 17.1 | EP | D1 | 645- polyploid Hybrid |
| 16.9 | EP | D1 | 646- polyploid Hybrid |
| 18.1 | EP | D1 | 649- polyploid Hybrid |
| 18.6 | EP | D1 | 664- polyploid Hybrid |
| 17.2 | EP | D1 | 683- polyploid Hybrid |
| 18.5 | EP | D1 | 685- polyploid Hybrid |

Thus, the present results show that the polyploid hybrid plant grain protein content was similar to that of the control plant. Thus, the genome multiplication protocol did not affect grain protein content in the polyploid hybrids common wheat plants.

TABLE 29
Crop Yield of Polyploid Hybrids Plant Compared to Control Plant

| Crop Yield | Crop | | | |
|---|---|---|---|---|
| Over | Yield | Ploidy | | |
| Control (%) | (Ton/Ha) | Level | Generation | Name |
| | 7.6 | 6n | F6+ | 3-control |
| | 7.5 | 6n | F6+ | 5-control |
| | 8.3 | 6n | F6+ | 18-control |
| 66 | 12.5 | EP | D1 | 645- polyploid Hybrid |
| 166 | 20.2 | EP | D1 | 646- polyploid Hybrid |
| 114 | 16.2 | EP | D1 | 649- polyploid Hybrid |
| 89 | 14.1 | EP | D1 | 664- polyploid Hybrid |
| 109 | 17.2 | EP | D1 | 683- polyploid Hybrid |
| 78 | 14.7 | EP | D1 | 685- polyploid Hybrid |

Thus, the polyploid hybrid common wheat plant having a partially or fully HI multiplied genome exhibited a significant increase in the crop yield of up to two hundreds percent compared to the control plant under the same developmental stage and growth conditions. Thus, the plants exhibited full seed set indicating that the enhanced polyploid (EP) plants had at least equivalent fertility as the control plants.

TABLE 30
Dry Matter Weight of Polyploid Hybrids
Plant Compared to Control Plant

| Dry Matter | Dry Matter | | | |
|---|---|---|---|---|
| Weight per | Weight | | | |
| plant Over | per plant | Ploidy | Gener- | |
| Control (%) | (Ton/Ha) | Level | ation | Name |
| | 162.6 | 6n | F6+ | 3-control |
| | 106.3 | 6n | F6+ | 5-control |
| | 206.5 | 6n | F6+ | 18-control |
| 49 | 242.6 | EP | D1 | 645- polyploid Hybrid |
| 153 | 410.5 | EP | D1 | 646- polyploid Hybrid |
| 91 | 310.0 | EP | D1 | 649- polyploid Hybrid |
| 103 | 215.2 | EP | D1 | 664- polyploid Hybrid |
| 87 | 386.0 | EP | D1 | 683- polyploid Hybrid |
| 16 | 239.0 | EP | D1 | 685- polyploid Hybrid |

Thus, the polyploid hybrid common wheat plant having a partially or fully multiplied genome demonstrated a significant increase in dry matter weight of over two fold compared to the control plant under the same developmental stage and growth conditions. Hence, the higher quantity of the dry matter weight is indicative of high bio-mass accumulation in the polyploid hybrid plants. In addition, these results indicate that the vigor and the heterosis effect are higher in the hybrid plants compared to control plants.

TABLE 31
Spike Data of Polyploid Hybrids
Compared to Female Control Lines

| No. Seeds per | Spike | Spike | Spike | |
|---|---|---|---|---|
| Spikelet | internodes | width | length | Name |
| 4.5 | 12 | 2.5 | 12.5 | 3-control |
| 3.5 | 12 | 2.0 | 12.0 | 32 polyploid Hybrid |
| 5.0 | 12 | 2.2 | 13.0 | 5 control |
| 4.5 | 13 | 2.2 | 13.5 | 40 polyploid Hybrid |

Thus, the present results showed that the spike length and width were not essentially affected by the genome multiplication protocol in the enhanced polyploid or by the hybridization crossing with common wheat plants compared to control. Similarly, the seeds number per spikelet and the spike internodes in the hybrids plants remained essentially the same with no statistically significant differences compared to the isogenic plant.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A common wheat plant having a partially or fully multiplied genome being at least as fertile as a hexaploid common wheat (Triticum aestivum L.) plant isogenic to said genomically multiplied common wheat plant when grown under the same conditions.

2. A hybrid plant having as a parental ancestor the plant of claim 1.

3. A hybrid common wheat plant having a partially or fully multiplied genome.

4. A planted field comprising the plant of claim 1.

5. A sown field comprising seeds of the plant of claim 1.

6. The plant of claim 1 being non-transgenic.

7. The plant of claim 1, wherein said fertility is exhibited at least on third generation of said hexaploid common wheat having said partially or fully multiplied genome.

8. The plant of claim 1 having a spike number at least as similar to that of said hexaploid common wheat (Triticum aestivum L.) plant under the same developmental stage and growth conditions.

9. The plant of claim 1 having a spikelet number at least as similar to that of said hexaploid common wheat (Triticum aestivum L.) plant under the same developmental stage and growth conditions.

10. The plant of claim 1, having a spike length at least as similar to that of said hexaploid common wheat (Triticum aestivum L.) plant under the same developmental stage and growth conditions.

11. The plant of claim 1, having a grain number per spikelet ratio at least as similar to that of said hexaploid common wheat (Triticum aestivum L.) plant under the same developmental stage and growth conditions.

12. The plant of claim 1, having a spike width at least as similar to that of said hexaploid common wheat (Triticum aestivum L.) plant under the same developmental stage and growth conditions.

13. The plant of claim 1, having a spike internode number at least as similar to that of said hexaploid common wheat (Triticum aestivum L.) plant under the same developmental stage and growth conditions.

14. The plant of claim 1, having a grain protein content at least as similar to that of said hexaploid common wheat (Triticum aestivum L.) plant under the same developmental stage and growth conditions.

15. The plant of claim 1, having a dry matter content at least as similar to that of said hexaploid common wheat (Triticum aestivum L.) plant under the same developmental stage and growth conditions.

16. The plant of claim 1, having a grain yield per growth area at least as similar to that of said hexaploid common wheat (Triticum aestivum L.) plant under the same developmental stage and growth conditions.

17. The plant of claim 1, having a total grain number per plant ratio at least as similar to that of said hexaploid common wheat (Triticum aestivum L.) plant under the same developmental stage and growth conditions.

18. The plant of claim 1, having a grain weight at least as similar to that of said hexaploid common wheat (Triticum aestivum L.) plant under the same developmental stage and growth conditions.

19. The plant of claim 1, having a grain yield per plant at least as similar to that of said hexaploid common wheat (Triticum aestivum L.) plant under the same developmental stage and growth conditions.

20. The plant of claim 1, having a rust tolerance higher than that of said hexaploid common wheat (Triticum aestivum L.) plant under the same developmental stage and growth conditions.

21. The plant of claim 1, having a total plant length similar or lower than that of said hexaploid common wheat (Triticum aestivum L.) plant under the same developmental stage and growth conditions.

22. The plant of claim 1, wherein said fertility is determined by at least one of:
number of seeds per plant;
seed set assay;
gamete fertility assay; and
acetocarmine pollen staining.

23-25. (canceled)

26. The plant of claim 1, capable of cross-breeding with a hexaploid or a tetraploid wheat.

27. (canceled)

28. The hybrid plant of claim 2, wherein having a Durum wheat as a second parental ancestor.

29. (canceled)

30. A plant part of the common wheat plant of claim 1.

31. A processed product of the plant or plant part of claim 1.

32-33. (canceled)

34. A meal produced from the plant or plant part of claim 1.

35. The plant part of claim 30 being a seed.

36. An isolated regenerable cell of the common wheat plant of claim 1.

37. The cell of claim 35, exhibiting genomic stability for at least 5 passages in culture.

38-39. (canceled)

40. A method of producing common wheat seeds, comprising self-breeding or cross-breeding the plant of claim 1.

41. A method of developing a hybrid plant using plant breeding techniques, the method comprising using the plant of claim 1 as a source of breeding material for self-breeding and/or cross-breeding.

42. A method of producing common wheat meal, the method comprising:
(a) harvesting grains of the common Triticum aestivum L. plant or plant part of claim 1; and
(b) processing said grains so as to produce the common Triticum aestivum L. meal.

43. A method of generating a common wheat having a partially or fully multiplied genome, the method comprising contacting the common wheat (Triticum aestivum L.) seed with a G2/M cell cycle inhibitor under a transiently applied magnetic field thereby generating the common wheat seed having a partially or fully multiplied genome.

44. The method of claim 43, wherein said G2/M cell cycle inhibitor comprises a microtubule polymerization inhibitor.

45. The method of claim 44, wherein said microtubule polymerization inhibitor is selected from the group consisting of colchicine, nocodazole, oryzaline, trifluraline and vinblastine sulphate.

46. The method of claim 43, further comprises sonicating said seed prior to contacting.

47. A sample of representative seeds of a common wheat plant having a partially or fully multiplied genome being at least as fertile as a hexaploid common wheat (Triticum aestivum L.) plant isogenic to said genomically multiplied common wheat plant when grown under the same conditions, wherein said sample has been deposited under the Budapest Treaty at the NCIMB under NCIMB 41972.

48. A sample of representative seeds of a common wheat plant having a partially or fully multiplied genome being at least as fertile as a hexaploid common wheat (Triticum aestivum L.) plant isogenic to said genomically multiplied common wheat plant when grown under the same conditions, wherein said sample of said common wheat plant having said partially or fully multiplied genome has been deposited under the Budapest Treaty at the NCIMB under NCIMB 41972.

49. The plant of claim 1 is not a genomically multiplied haploid plant.

50. The plant of claim 1, wherein said fertility is exhibited at least on second generation following genomic multiplication of said isogenic hexaploid common wheat.

51. The plant of claim 1, exhibiting genetic stability for at least three generations.
